SEQUENCE LISTING (5 sequences)

SEQ ID NO: 1 - DNA, 19 bp, Artificial Sequence
  Synthetic
ggaactactggtgaactat

SEQ ID NO: 2 - DNA, 19 bp, Artificial Sequence
  Synthetic
agagcgaacttgtagttga

SEQ ID NO: 3 - DNA, 19 bp, Artificial Sequence
  Synthetic
ccaacgacctgggcaaaaa

SEQ ID NO: 4 - DNA, 21 bp, Artificial Sequence
  Synthetic
ggaacuacuggugaacuautt

SEQ ID NO: 5 - DNA, 21 bp, Artificial Sequence
  Synthetic
auaguucaccaguaguucctg

(19) United States
(12) Patent Application Publication
(10) Pub. No.: US 2018/0017561 A1
(43) Pub. Date: Jan. 18, 2018

(54) TH17-PRONE CD146+CCR5+ T-CELL POPULATION AS AN EARLY MARKER OF INTESTINAL GRAFT-VERSUS-HOST DISEASE

(71) Applicant: Indiana University Research and Technology Corporation, Indianapolis, IN (US)

(72) Inventor: Sophie Peczesny, Indianapolis, IN (US)

(21) Appl. No.: 15/546,979

(22) PCT Filed: Jan. 28, 2016
(86) PCT No.: PCT/US16/15352

§ 371 Date: Jul. 27, 2017

Related U.S. Application Data
(60) Provisional application No. 62/109,264, filed on Jan. 29, 2015.

(51) Int. Cl. G01N 33/569; G01N 33/567; A61K 49/00

(52) U.S. Cl. CPC G01N 33/56972; A61K 49/0058; G01N 33/567; A61K 49/0043; G01N 2800/245; G01N 2800/52; G01N 2333/70521

(57) ABSTRACT

Th17-prone CD146⁺CCR5⁺ T-cell population as an early biomarker of intestinal graft-versus-host disease and its use for determining prognosis of intestinal GVHD before the clinical signs are apparent are disclosed.

### CROSS-REFERENCE TO RELATED APPLICATIONS

This application claims priority to International Application Number PCT/US2016/015352, filed on 28 January, 2016, which claims priority to U.S. Provisional Patent Application Ser. No. 62/109,264, filed Jan. 29, 2015, both of which are hereby incorporated by reference in their entireties.

### STATEMENT OF GOVERNMENT SUPPORT

This invention was made with government support under CA168814 awarded by the National Institutes of Health. The Government has certain rights in the invention.

### INCORPORATION OF SEQUENCE LISTING

A paper copy of the Sequence Listing and a computer readable form of the sequence containing the file named IURTC 2015-101-03_ST25.txt”, which is 1,321 bytes in size (as measured in MICROSOFT WINDOWS EXPLORER), are provided herein and are herein incorporated by reference. This Sequence Listing consists of SEQ ID NOs:1-5.

### BACKGROUND OF THE DISCLOSURE

The present disclosure relates generally to biomarkers of graft-versus-host disease. More particularly, the present disclosure relates to a Th17-prone CD146⁺CCR5⁺ T-cell population as an early biomarker of intestinal graft-versus-host disease and their use for determining treatment responsiveness.

Allogeneic hematopoietic stem cell transplantation (HSCT) is a potential curative therapy for cancers of the blood and bone marrow. Use of HSCT has increased as new techniques have allowed for transplantation in patients who previously would not have been considered HSCT candidates. Approximately 30,000 allogeneic HSCTs will be performed worldwide in 2020; however, the efficacy of this procedure has been impeded by acute graft-versus-host disease (aGVHD). GVHD is a common complication following a bone marrow transplant from a donor. It occurs after transplant, when the donor's lymphocytes recognize parts of the patient's body as foreign. During this process, molecules (including cytokines and their receptors) are released that may damage certain body tissues, including the gut, liver and skin. The diagnosis of GVHD currently relies on clinical symptoms and biopsies of the main target organs: skin, liver and gastrointestinal tract (GI). Some of the main effects can include red skin rash, diarrhea, sometimes with blood, and yellow jaundice. GVHD can be serious, with complications that range from mild to life threatening, even death, and often requires admission to the hospital for treatment. The standard preventive measures for aGVHD is a combination of two drugs that suppress the immune system, such as tacrolimus (or cyclosporine) and Methotrexate. Despite the use of this regimen, aGVHD is expected to develop in about 50% of transplant recipients. The standard treatment for acute GVHD includes steroids, in addition to the above mentioned prophylaxis immunosuppressive medications.

Patients who develop steroid refractory aGVHD have poor prognosis with expected 1 year mortality of up to 90%. In addition, a significant proportion of patients require re-escalation of steroids or addition of second-line therapy due to recurrence of aGVHD or steroid intolerance. This high mortality rate is not only due to aGVHD refractoriness, but also to the toxicity of the immunosuppressive treatment that inhibits the pathogen specific immunity leading to opportunistic infections and the leukemia specific immunity leading to tumor relapses, all causes of outstanding morbidity and mortality in this already fragile population.

In the past 30 years, therapeutic approaches for aGVHD have largely been limited to targeting effector cells nonspecifically. To date, the only two options to mitigate aGVHD are (1) aGVHD prophylaxis, which, if increased, might increase the risk of opportunistic infections and malignancies relapses, and (2) aGVHD treatment, which has a failure rate of approximately 50%. There are no validated laboratory tests to predict the risk of developing aGVHD and subsequent patient survival. One major reason for this is the lack of available biomarkers with sufficient sensitivity and reasonable specificity. The absence of validated biomarkers for aGVHD is further due to the complex pathology of aGVHD, which involves both soluble and cellular factors.

Based on the foregoing, there is a need in the art for a biomarker, or a panel of biomarkers, of GVHD resistance that can predict GVHD before the clinical signs emerge. More particularly, it would be advantageous if a biomarker, or a panel of biomarkers, could predict early risk for aGVHD of the gastrointestinal tract (GI GVHD), which is the aGVHD target organ most associated with non-relapse mortality (NRM) following HSCT.

### SUMMARY OF THE DISCLOSURE

The present disclosure relates generally to biomarkers of graft-versus-host disease. More particularly, the present disclosure relates to the use of a Th17-prone CD146⁺CCR5⁺ T-cell population as an early biomarker of intestinal graft-versus-host disease and a guide for starting treatment in a preemptive manner to avert the development of steroid refractory aGVHD. Additionally, by determining biomarker thresholds with the goal of reaching 80% sensitivity for aGVHD development and demonstrating the predictive validity for subsequent six-month (6 m) post-transplant non-relapse mortality (NRM) and 6-month freedom from treatment failure (6 m FFTF) in this population, the biomarkers can be used to identify a high risk population for therapy-resistant aGVHD.

Accordingly, in one aspect, the present disclosure is directed to a method of diagnosing or of aiding diagnosis of acute graft-versus-host disease (aGVHD) in a subject receiving hematopoietic stem cell transplantation (HSCT), the method comprising: measuring in a biological sample from the subject receiving HSCT the CD146⁺CCR5⁺ T cell frequency; and comparing the CD146⁺CCR5⁺ T cell frequency of the subject receiving HSCT to the CD146⁺CCR5⁺ T cell frequency of a control subject receiving HSCT without aGVHD; wherein an elevated CD146⁺CCR5⁺ T cell frequency of the subject receiving HSCT compared to the CD146⁺CCR5⁺ T cell frequency of the control subject is indicative of aGVHD.

In another aspect, the present disclosure is directed to a method of prognosing or of aiding prognosis of acute graft-versus-host disease (aGVHD) in a subject receiving hematopoietic stem cell transplantation (HSCT), the method comprising: measuring in a biological sample from the subject receiving HSCT the CD146⁺CCR5⁺ T cell frequency; and comparing the CD146⁺CCR5⁺ T cell frequency of the subject receiving HSCT to the CD146⁺CCR5⁺ T cell frequency of a control subject receiving HSCT without aGVHD; wherein an elevated CD146⁺CCR5⁺ T cell frequency of the subject receiving HSCT compared to the CD146⁺CCR5⁺ T cell frequency of the control subject is indicative of aGVHD.

In another aspect, the present disclosure is directed to a method of measuring treatment efficacy in an aGVHD subject, the method comprising: measuring in a first biological sample from the subject a baseline CD146⁺CCR5⁺ T cell frequency; administering a treatment for aGVHD; and measuring in a second biological sample from the subject a post-treatment CD146⁺CCR5⁺ T cell frequency; wherein a post-treatment CD146⁺CCR5⁺ T cell frequency that is equal to or greater than the baseline CD146⁺CCR5⁺ T cell frequency is indicative of treatment inefficacy.

This aspect of the present disclosure also provides a method of measuring treatment efficacy in an aGVHD subject, the method comprising: measuring in a first biological sample from the subject a baseline CD146⁺CCR5⁺ T cell frequency; and measuring in a second biological sample from the subject, who has received a treatment for aGVHD, a post-treatment CD146⁺CCR5⁺ T cell frequency; wherein a post-treatment CD146⁺CCR5⁺ T cell frequency that is equal to or greater than the baseline CD146⁺CCR5⁺ T cell frequency is indicative of treatment inefficacy.

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1 depicts a gating strategy for conventional T cells (Tconvs) and regulatory T cells (Tregs).

FIGS. 2A & 2B depicts a CD146CCR5 CD4 T-cell population in allogeneic HCT patients at GVHD onset and prior GVHD onset. FIG. 2A depicts CD146CCR5 CD4 T cells measured in the conventional T cell gate (CD4+CD25loCD127+). FIG. 2B is a graph depicting CD146CCR5 T-cell frequencies in healthy donors (HD), autotransplant patients (Auto) and allogeneic patients (all others including skin first with subsequent GI-GVHD) measured by flow cytometry. N and median post-HCT onset of signs or samples are shown below the graphs. The data are shown as mean±standard error of the mean (SEM). Unpaired t-test, significant at p<0.05.

FIG. 2C depicts CD146CCR5 T-cell frequency in paired samples prior to GVHD onset in GI-GVHD patients (n=31) with a median interval of 14 days between the first measurement and measurement at onset of disease.

FIGS. 3A & 3B depict absolute lymphocyte counts and CD146CCR5 T-cell counts in allogeneic patients following HCT as measured by multicolor flow cytometry. Data represent mean±standard error of the mean (SEM), and differences were analyzed using unpaired t-tests.

FIGS. 4A & 4B depict CD146+CCR5+ T convs frequency on CD4+ T cells stratified by onset of GI GVHD stage (FIG. 4A) and maximum GI GVHD stage (FIG. 4B). Stage 0 at onset includes isolated skin GVHD patients, non-GVHD enteritis and patients without GVHD (unpaired t-test, significance for p<0.05).

FIGS. 5A & 5B depict frequencies of CD146 T cells and CCR5 T cells in HCT patients. Frequencies of (FIG. 5A) CD146 T cells and (FIG. 5B) CCR5 T cells in allogeneic patients following HCT. Data represent mean±SEM, and differences were analyzed using unpaired t tests.

FIG. 6 depicts CD146 and CCR5 expression on CD4 and CD8 T cells and monocytes in healthy donors. CD146 and CCR5 staining on CD4 cells, CD8 cells, and CD14 monocytes from PBs prepared from healthy donors using antibodies against CD146 (clone P1H12, eBioscience) and CCR5 (clone 2D7, BD Biosciences).

FIG. 7 depicts receiver operating characteristic (ROC) curve comparing GI-GVHD versus non-GVHD enteritis HCT patients. ROC curve of CD146CCR5 T cells frequency comparing GI GVHD (n=71) versus non GVHD enteritis (n=33), Area Under the Curve: AUC=0.84.

FIG. 8 depicts six-month non-relapse mortality in allogeneic HCT patients with symptoms divided by low and high CD146CCR5 T-cell frequencies. Low and high CD146CCR5 T cell frequencies and outcomes in all patients with symptoms (GI-GVHD, non-GVHD enteritis, skin only, and skin first GVHD, n=166). High-risk group (CD146CCR5 T cell frequency ≧2.3%) and low-risk group (CD146CCR5 T-cell frequency <2.3%), p=0.0001.

FIGS. 9A-9C depict CD146CCR5 T-cell frequency by Reg3α and ST2 plasma concentrations, GI symptom localization, and GI-GVHD histologic grade. FIG. 9A shows the correlation of Reg3α and ST2 plasma concentrations with CD146CCR5 T-cell frequency in patients with GI-GVHD. The squared Pearson correlation coefficient was used. FIG. 9B depicts CD146CCR5 T-cell frequency classified by GI symptom localization, unpaired t-tests. FIG. 9C depicts CD146CCR5 T-cell frequency stratified by GI-GVHD histologic grade, unpaired t-test.

FIG. 10 depicts clonality of the sorted CD146+CCR5+ and CD146−CCR5− T cells as determined by deep sequencing of TCRβ regions in the T-cell populations (n=3, t test).

FIGS. 11A & 11B depict the TH17-committed CD146 CCR5 T cell population. FIG. 11A depicts differential transcriptomes in sorted-CD146CCR5 T cells versus T cells excluding this population using NANOSTRING® technology. Representative genes upregulated in the double positive population. FIG. 11B depicts intracellular staining for Th17-transcription factor RORC and its corresponding cytokine IL-17 in the same subsets in patients' samples (n=35 and 41, respectively).

FIG. 12 depicts expression of Th1-transcription factor TBET in CD146CCR5 T-cell subset versus T cells excluding this population in GI-GVHD patients. Intracellular staining for Th1-transcription factor TBET in GI-GVHD patients' samples (n=35), unpaired t test.

FIGS. 13A & 13B depict CD146CCR5 T-cell effector memory phenotype. FIG. 13A shows representative plots showing the expression of CD45RA and CCR7 on HCT patient T cells gated on the CD146+CCR5+ T-cell population or excluding this population, and FIG. 13B is a bar graph depicting mean±SEM values for the frequencies of CD45RA-CCR7− cells in the same subsets in patients' samples (n=50).

FIGS. 14A & 14B depict in vitro Th1 or Th17 differentiation of naïve T cells with anti-CD3/anti-CD28 bead stimulation. FIG. 14A shows representative plots of CD146 expression in CD4 T cells, and FIG. 14B is a bar graph depicting mean±SEM values for frequency of CD146 in the two conditions (n=16).

FIGS. 15A & 15B depict CD146CCR5 T-cell generation in allogeneic mixed lymphocyte reactions (MLRs). FIG. 15A are representative plots showing the expression of CD146 and CCR5 on T cells from autologous and allogeneic MLRs. FIG. 15B shows the mean±SEM of the frequency of CD146CCR5 T cells pooled from 12 independent experiments, and differences were analyzed using unpaired t test.

FIGS. 16A & 16B depict in vitro Th1 or Th17 differentiation of naïve T cells with anti-CD3/anti-CD28 or anti-CD3/anti-ICOS bead stimulation, respectively. FIG. 16A are representative plots of CD146 and CCR5 expression in CD4 T cells, and FIG. 16B is a bar graph depicting mean±SEM values for frequency of CD146CCR5 T cells in the four conditions (n=4; bottom panel). Unpaired t test, significant at p<0.05.

FIGS. 17A & 17B depict in vitro Th1 or Th17 differentiation of naïve T cells with anti-CD3/anti-CD28 or anti-CD3/anti-ICOS bead stimulation, respectively. FIG. 17A are representative plots of IL-17 and IFN-γ coexpression in CD4 T cells, and FIG. 17B is a bar graph depicting mean±SEM values for frequency of IL-17IFN-y T cells in the four conditions (n=7). Unpaired t test, significant at p<0.05.

FIG. 18 depicts Th17 commitment of sorted CD146+ versus CD146− Tconvs. Mature CD4+CD146− and CD4+CD146+ T cells were isolated from total CD4 T cells and cultured under Th1 or Th17 polarizing conditions for 7 days. Anti-CD3/anti-CD28- or anti-CD3/anti-ICOS-coated Dynabeads were added on day 0 (D0) to activate the cells. The frequency of IL-17A+IFN-γ+ Tconvs on CD4+ T cells was measured by flow cytometry. (Statistical data were pooled from 4 independent experiments, t test, mean±SEM).

FIGS. 19A & 19B depict Th17-associated markers in ICOS stimulated Th17 cells and CD146CCR5 T cells. FIG. 19A shows frequencies of CD161-, IL-23R- or CXCR6-expressing T cells after CD28 and ICOS stimulation with Th1 or Th17 differentiation. Naïve CD4 T cells were isolated from healthy donor PB cells and cultured in the presence of anti-CD3/anti-CD28 or anti-CD3/anti-ICOS beads under Th1 or Th17 polarizing conditions. After 7 days, the frequencies of CD161-, IL-23R- or CXCR6-expressing T cells were measured by flow cytometry. Summary data were pooled from 3 independent experiments, presented as mean±SEM, and analyzed by one-way ANOVA. FIG. 19B shows frequencies of Th17-associated markers in the CD146CCR5 double-positive cells as compared to in T cells excluding this population. Conventional CD4 T cells were stimulated with anti-CD3/anti-ICOS beads for 7 days under Th17-polarizing conditions and analyzed by flow cytometry. Summary data are presented as mean±SEM and were analyzed by paired t test (n=3 for IL-17IL-22; n=4 for IL-17GM-CSF, CCR4, and CCR6; n=5 for IL23R; n=6 for IL-17IFN-γ, CD161, and CXCR6).

FIGS. 20A & 20B depict endothelial CD146 expression in GI-GVHD colonic biopsies, transmigration of CD4 T-cell subsets through human microvascular endothelial cells (HMVECs) and with CD146 knockdown and control, and chemotaxis of CD146CCR5 T cells towards CCL14. FIG. 20A depicts immunohistochemical analysis of colonic biopsies taken at onset of symptoms from non-GVHD enteritis patients (left panel), and GI-GVHD patients (right panel) for CD146 expression (magnification×200). FIG. 20B is a bar graph showing mean±SEM values for CD146+ vessel counts×10, unpaired t test from non-GVHD enteritis patients (n=10) and GI-GVHD patients (n=18).

FIGS. 21A-21C depict transmigration through HMVECs of CD146− and CD146+ T cells sorted from fresh PB cells. Representative flow cytometric histograms showing the efficiency of CD146 knockdown in Th17 cells with CD146 shRNA1 and CD146 shRNA2. Isotype control staining and CD146 staining of cells with the control shRNA and CD146 shRNA. Bars show mean±SEM values for percentage of transmigrated CD4 T cells (n=6 for fresh CD146− and CD146+ T cells groups, n=5 for Th1 and Th17 groups, n=5 for CD146 shRNA1 and shRNA control groups, and n=8 for CD146 shRNA2 and shRNA control groups, unpaired t test).

FIGS. 22A-22C depict transmigration through HMVECs of Th1 and Th17 differentiated cells, Th17 differentiated cells with CD146 knockdown via control shRNA, CD146 shRNA1 or CD146 shRNA2. Representative flow cytometric histograms showing the efficiency of CD146 knockdown in Th17 cells with CD146 shRNA1 and CD146 shRNA2. Isotype control staining and CD146 staining of cells with the control shRNA and CD146 shRNA. Bars show mean±SEM values for percentage of transmigrated CD4 T cells (n=6 for fresh CD146− and CD146+ T cells groups, n=5 for Th1 and Th17 groups, n=5 for CD146 shRNA1 and shRNA control groups, and n=8 for CD146 shRNA2 and shRNA control groups, unpaired t test).

FIG. 23 depicts CCR5-mediated chemotaxis of sorted CD146CCR5 T cells toward CCL14 and CCL5. The sorted double positive cells were pre-treated with CCR5 antagonist maraviroc (MV) or vesicle control and added to the top well of a transwell chamber. CCL14 or a mix of CCL14 and CCL5 was added to the bottom well. Chemotaxis is expressed as a ratio of the numbers of cells that migrated to the bottom wells containing CCL14 or CCL14/CCL5 cocktail and medium alone. Data are presented as mean±SEM values (n=3, paired t test).

FIGS. 24A-24D depict donor human T cells with CD146 knockdown via CD146 shRNA in a xenogeneic GVHD model. FIG. 24A show sublethally irradiated NSG mice transplanted with human CD4 T cells transduced with CD146 shRNA or control shRNA lentivirus and analyzed for body weight loss. Data are pooled from 8 mice for the control shRNA group and 9 mice for the CD146 shRNA group from 2 independent experiments. FIG. 24B depicts Kaplan-Meier survival curves. Data are pooled from 30 control mice and 27 mice in the CD146 group from five independent experiments (3 with CD146 shRNA1 and 2 with CD146 shRNA2), log-rank test. FIG. 24C depicts human CD4 T-cell engraftment in spleen. FIG. 24D depicts CD146CCR5 T-cell frequency in the gut. Mice were analyzed between days 30-45 after transplantation (FIGS. 24C & 24D). Representative flow cytometric plots are shown at the top, and bar graphs showing mean±SEM values at the bottom (unpaired t test).

FIG. 24E depicts absolute counts of intestinal CD146CCR5 T cells in xenogeneic GVHD with control shRNA or CD146 shRNA knockdown. Sublethally irradiated NSG mice were transplanted with human CD4 T cells transduced with CD146 shRNA or control shRNA lentivirus. Then, the absolute number of CD146CCR5 T cells were analyzed in the gut. Mice were analyzed between days 30-45 after transplantation. n=10, unpaired t test.

FIGS. 24F-24M depicts Th17 cells coexpressing IL-17 and IFN-γ in the spleen. Mice were analyzed between days 30-45 after transplantation (FIG. 24F). Representative flow cytometric plots are shown at the top, and bar graphs showing mean±SEM values at the bottom (unpaired t test). The data in FIG. 24F was pooled from 5 mice of the control group and 4 mice of the CD146 group from 2 independent experiments. For weight loss, unpaired t tests, *p<0.05, **p<0.01. (FIGS. 24G & 24M). The percent body weight loss of NSG mice transplanted with CD146 shRNA or control shRNA lentivirus and treated with maroviroc or vesicle control from independent experiments (unpaired t test). FIG. 24H depicts human T-cell infiltration in the gut of mice receiving transduced CD4 T cells and maraviroc or vesicle control. Mice were analyzed between 26-39 days. The data were pooled from 4 mice per group and from 2 independent experiments (ANOVA). FIGS. 24I-24L show pathology indexes of intestine (FIG. 24I), liver (FIG. 24J), skin (FIG. 24K), and lung (FIG. 24L) for NSG mice transplanted with CD146 shRNA or control shRNA and treated with maraviroc or vehicle. Mice were analyzed between 26-38 days (n=6, t test).

FIGS. 25A-25H depict CD146CCR5 regulatory T cells (Tregs) in allogeneic HCT patients at GVHD onset and ICOS-induced CD146CCR5 Tregs. FIG. 25A shows CD25+CD127-FOXP3+ Treg frequencies, and FIG. 25B shows CD146CCR5 Treg frequencies in patients with GI-GVHD, without GVHD, with non-GVHD enteritis, and with skin only GVHD as measured by flow cytometry. N and median post-HCT onset of signs or samples are shown below the graphs. The data are shown as mean±standard error of the mean (SEM). Unpaired t test, significant at p<0.05. FIG. 25C shows differential transcriptomes in sorted-CD146CCR5 Tregs versus Tregs excluding this population using Nanostring® technology. Representative genes upregulated in the double-positive population. FIG. 25D depict intracellular staining for IFN-γ and IL-17 in the CD146+CCR5-, CD146−CCR5+, and CD146+CCR5+ subsets of Tregs from healthy donors. FIG. 25E show PD1 expression on CD146CCR5 Tregs and Tregs excluding this population from 17 GI-GVHD patients. Unpaired t test. FIG. 25F depict in vitro stimulation of CD25+CD127− Tregs with anti-CD3/anti-CD28 or anti-CD3/anti-ICOS beads. Representative plots of CD146 and CCR5 expression in Tregs (top panel), and bar graph depicting mean±SEM values for frequency of CD146CCR5 Tregs in the two conditions (n=3; bottom panel). Unpaired t test. FIG. 25G show that Tregs were stimulated with anti-CD3/anti-CD28 or anti-CD3/anti-ICOS beads and analyzed for intracellular cytokine production. Representative plots of IL-17 and IFN-y coexpression in Tregs (top panel) and bar graph depicting mean+SEM values for frequency of IL-17/IFN-γ T cells in the two conditions (n=3; bottom panel). Unpaired t test. FIG. 25H depicts mean fluorescent intensity of FOXP3 in Tregs after anti-CD3/anti-CD28 or anti-CD3/anti-ICOS bead stimulation. Representative plots of FOXP3 expression in Tregs (top panel), and a bar graph depicting mean±SEM values for normalized MFI of the Tregs in the two conditions (n=4; bottom panel). Unpaired t test.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

Unless defined otherwise, all technical and scientific terms used herein have the same meaning as commonly understood by one of ordinary skill in the art to which the disclosure belongs. Although any methods and materials similar to or equivalent to those described herein may be used in the practice or testing of the present disclosure, the preferred materials and methods are described below.

### A. Definitions

As used herein, the term “biomarker” refers to a characteristic that is objectively measured and evaluated as an indicator of normal biologic processes, pathogenic processes, or biological responses to a therapeutic intervention. Biomarkers include cellular-based, DNA-based, RNA-based and protein-based molecular markers.

As used herein, the term “diagnosis” refers to the identification or classification of a molecular or pathological state, disease or condition. For example, “diagnosis” can refer to identification of a particular type of a condition (such as acute graft-versus host disease (“aGVHD”)).

As used herein, the term “aiding diagnosis” refers to methods that assist in making a clinical determination regarding the presence, or nature, of a particular type of symptom or condition (such as aGVHD). For example, a method of aiding diagnosis of a condition (such as aGVHD) can include measuring the frequency of certain cell populations in a biological sample from a subject.

As used herein, the term “prognosis” refers to the categorization of subjects by degree of risk for a disease (such as aGVHD) or progression of such disease. A “prognostic marker” refers to an assay that categorizes subjects by degree of risk for disease occurrence or progression.

As used herein, the term “sample” refers to a composition that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized. A “control sample” refers to any sample obtained from a healthy individual, a patient receiving uncomplicated allogenic transplant, and a patient with non-GVHD enteritis. A “tissue” or “cell sample” refers to a collection of similar cells obtained from a tissue of a subject or patient. The source of the tissue or cell sample may be blood or any blood constituents (e.g., whole blood, plasma, serum) from the subject. The tissue sample can also be primary or cultured cells or cell lines. Optionally, the tissue or cell sample is obtained from a disease tissue/organ. The tissue sample can contain compounds which are not naturally intermixed with the tissue in nature such as preservatives, anticoagulants, buffers, fixatives, nutrients, antibiotics, and the like.

The term “subject” is used interchangeably herein with “patient” to refer to an individual to be treated. The subject is a mammal (e.g., human, non-human primate, rat, mouse, cow, horse, pig, sheep, goat, dog, cat, etc.). The subject can be a clinical patient, a clinical trial volunteer, an experimental animal, etc. The subject can be suspected of having or at risk for having a condition (such as aGVHD) or be diagnosed with a condition (such as aGVHD). According to one embodiment, the subject to be treated according to this disclosure is a human.

As used herein, “treating”, “treatment” and “alleviation” refer to measures, wherein the object is to prevent or slow down (lessen) the targeted pathologic condition or disorder or relieve some of the symptoms of the disorder. Those in need of treatment can include those already with the disorder as well as those prone to have the disorder, those at risk for having the disorder and those in whom the disorder is to be prevented.

As used herein, “elevated levels” refers to an increased frequency of a cell population and/or expression of a mRNA or a protein in a subject (e.g., a patient suspected of having or diagnosed as having aGVHD) relative to a control, such as subject or subjects who are not suffering from aGVHD. In particular embodiments, the control subject can be a healthy individual, a patient receiving uncomplicated allogenic transplant (that is, a patient receiving allogenic transplant without severe complications, e.g., aGVHD, idiopathic pneumonia syndrome, veno-occlusive disease, sepsis and the like, and a patient with non-GVHD enteritis).

### B. Methods of Prognosing

In one embodiment, the present disclosure is directed to a method of prognosing or of aiding in the prognosis of aGVHD in a subject receiving hematopoietic stem cell transplantation (HSCT). The method includes: measuring in a biological sample from the subject receiving HSCT a CD146⁺CCR5⁺ T cell frequency; and comparing the CD146⁺CCR5⁺ T cell frequency of the subject receiving HSCT to a CD146⁺CCR5⁺ T cell frequency of a control subject not receiving HSCT; wherein an elevated T cell frequency of the subject receiving HSCT compared to the T cell frequency of the control subject is indicative of aGVHD.

In another embodiment, the present disclosure is directed to a method of prognosing or of aiding in the prognosis of aGVHD in a subject receiving hematopoietic stem cell transplantation (HSCT). The method includes: measuring in a biological sample from the subject receiving HSCT a CD146⁺CCR5⁺ T cell frequency; contacting the sample with an agent that specifically binds to a cell surface marker selected from CD146 and CCR5 cell surface markers to form a complex between the agent and the cell surface marker; detecting the complex to determine CD146⁺CCR5⁺ T cell frequency; and comparing the CD146⁺CCR5⁺ T cell frequency of the subject receiving HSCT to a CD146⁺CCR5⁺ T cell frequency of a control subject receiving HSCT without aGVHD; wherein an elevated T cell frequency of the subject receiving HSCT compared to the T cell frequency of the control subject is indicative of aGVHD.

Suitably, the methods of prognosing as described herein are used prior to the onset of clinical symptoms of aGVHD. In some embodiments, the methods are used to prognose a subject 7 days, even 14 days, or even 19 days or more prior to the onset of clinical symptoms.

In suitable embodiments, the subject receiving HSCT has or is susceptible to having gastrointestinal GVHD (GI GVHD). As used herein “susceptible to” refers to a subject that has received HSCT.

The CD146⁺CCR5⁺ T cell frequency in the biological sample(s) can be measured using any methods known in the art. Exemplary suitable methods for measuring the CD146⁺CCR5⁺ T cell frequency in the samples include immunohistochemistry, flow cytometry, mass cytometry (CYTOF), transmigration assay, and combinations thereof.

In some embodiments, the methods further include measuring the plasma suppressor of tumorigenicity (ST2) level in the subject receiving HSCT; and comparing the ST2 level of the subject receiving HSCT to the ST2 levels of the control subject wherein an elevated ST2 level of the subject receiving HSCT compared to the ST2 level of the control subject is further indicative of aGVHD.

### C. Methods of Diagnosing

In another embodiment, the present disclosure is directed to a method for diagnosing acute graft-versus-host disease (aGVHD) in a subject receiving hematopoietic stem cell transplantation (HSCT). The method includes: measuring in a biological sample from the subject receiving HSCT a CD146⁺CCR5⁺ T cell frequency; and comparing the CD146⁺CCR5⁺ T cell frequency of the subject receiving HSCT to a CD146⁺CCR5⁺ T cell frequency of a control subject not receiving HSCT; wherein an elevated T cell frequency of the subject receiving HSCT compared to the T cell frequency of the control subject is indicative of aGVHD.

In yet another embodiment, the present disclosure is directed to a method for diagnosing acute graft-versus-host disease (aGVHD) in a subject receiving hematopoietic stem cell transplantation (HSCT). The method includes: measuring in a biological sample from the subject receiving HSCT a CD146+CCR5+ T cell frequency; contacting the biological sample with an agent that specifically binds to a cell surface marker selected from CD146 and CCR5 cell surface markers to form a complex between the agent and the cell surface marker; detecting the complex to determine CD146⁺CCR5⁺ T cell frequency; and comparing the CD146⁺CCR5⁺ T cell frequency of the subject receiving HSCT to a CD146⁺CCR5⁺ T cell frequency of a control subject not receiving HSCT; wherein an elevated T cell frequency of the subject receiving HSCT compared to the T cell frequency of the control subject is indicative of aGVHD.

Suitably, the methods of diagnosing as described herein are used prior to the onset of clinical symptoms of aGVHD. In some embodiments, the methods are used to prognose a subject 7 days, even 14 days, or even 19 days or more prior to the onset of clinical symptoms.

In suitable embodiments, the subject receiving HSCT has or is susceptible to having gastrointestinal GVHD (GI GVHD).

The CD146⁺CCR5⁺ T cell frequency in the biological sample(s) can be measured using any methods known in the art. Exemplary suitable methods for measuring the CD146⁺CCR5⁺ T cell frequency in the samples include immunohistochemistry, flow cytometry, transmigration assay, and combinations thereof.

In some embodiments, the methods further include measuring the plasma suppressor of tumorigenicity (ST2) level in the subject receiving HSCT; and comparing the ST2 level of the subject receiving HSCT to the ST2 levels of the control subject wherein an elevated ST2 level of the subject receiving HSCT compared to the ST2 level of the control subject is further indicative of aGVHD.

### D. Methods of Measuring Treatment Efficacy

In another embodiment, the present disclosure is directed to a method of measuring treatment efficacy in an aGVHD subject. The method includes: measuring in a first biological sample from the subject a baseline CD146+CCR5+ T cell frequency; administering a treatment for aGVHD; and measuring in a second biological sample from the subject a post-treatment CD146+CCR5+ T cell frequency; wherein a post-treatment CD146+CCR5+ T cell frequency that is equal to or greater than the baseline CD146+CCR5+ T cell frequency is indicative of treatment inefficacy. Alternatively, the method in accordance with this embodiment may include: measuring in a first biological sample from the subject a baseline CD146⁺CCR5⁺ T cell frequency; and measuring in a second biological sample from the subject, who has received a treatment for aGVHD, a post-treatment CD146⁺CCR5⁺ T cell frequency; wherein a post-treatment CD146⁺CCR5⁺ T cell frequency that is equal to or greater than the baseline CD146⁺CCR5⁺ T cell frequency is indicative of treatment inefficacy.

In yet another embodiment, the present disclosure is directed to a method of measuring treatment efficacy in an aGVHD subject. The method includes: measuring in a first biological sample from the subject a baseline CD146+CCR5+ T cell frequency; administering a treatment for aGVHD; and measuring in a second biological sample from the subject a post-treatment CD146+CCR5+ T cell frequency; wherein a post-treatment CD146+CCR5+ T cell frequency that less than the baseline CD146+CCR5+ T cell frequency is indicative of treatment efficacy. Alternatively, the method in accordance with this embodiment of the invention may include: measuring in a first biological sample from the subject a baseline CD146⁺CCR5⁺ T cell frequency; and measuring in a second biological sample from the subject, who has received a treatment for aGVHD, a post-treatment CD146⁺CCR5⁺ T cell frequency; wherein a post-treatment CD146⁺CCR5⁺ T cell frequency that less than the baseline CD146⁺CCR5⁺ T cell frequency is indicative of treatment efficacy.

In suitable embodiments, the subject has or is susceptible to having gastrointestinal GVHD (GI GVHD).

The CD146⁺CCR5⁺ T cell frequency in a biological sample can be measured using any methods known in the art. Exemplary suitable methods for measuring the CD146⁺CCR5⁺ T cell frequency in the samples include immunohistochemistry, flow cytometry, transmigration assay, and combinations thereof.

Typically, the treatment for aGVHD can be any treatment known in the art. Suitable treatments for aGVHD include immunosuppressant agents and known therapeutic treatments such as cyclosporine, tacrolimus (also known as FK-506 or Fujimycin), methotrexate, mycophenoate, mofetil, antithymocyte globulin (ATG), monoclonal antibodies (e.g., anti-CD3, -CD5, and -IL-2 antibodies, anti-CD20 (rituximab), and alemtuzumab (Campath)), anti-TNF drugs (e.g., etanercept (ENBREL®, infliximab, adlimumab), lymphocyte immune globulin (ATGAM®), sirolimus, ustekinumab, extracorporeal photophoresis (ECP), anti-CD3 drugs (e.g., Visilizumab and OKT3), anti-CD5 drugs and anti-IL-2 (CD25) drugs (inolimomab, basiliximab, daclizumab, and denileukin diftitox), anti-CD147 drugs (e.g., Alefacept), anti-IL-1R drugs (e.g., Anakinra), mesenchymal stem cells, regulatory T cells, and combinations thereof. In some particularly suitable embodiments, steroids can also be used as treatment agents. Suitable steroids for treating aGVHD include, for example, corticosteroids (e.g., prednisone, prednisolone, methylprednisolone, and combinations thereof). The list of treatments provided herein above is not meant to be limiting as a person skilled in the art is aware of the many available treatment options for GVHD, aGVHD, and GI GVHD.

### E. Biological Sample

The biological sample(s) used in the methods of the present disclosure can be obtained using certain methods known to those skilled in the art. Biological samples may be obtained from vertebrate animals, and in particular, mammals, and more particularly, humans. In certain instances, a biological sample is blood, plasma, or serum. By screening such body samples, a prognosis, a diagnosis, and/or treatment efficacy can be achieved for aGVHD.

### EXAMPLES

### Example 1

A proteomic comparison for determining the ability of biomarkers to predict responsiveness to the treatment and mortality of aGVHD was conducted as described in US Publication No. 2013/0115232, published May 9, 2013, which is incorporated herein by reference to the extent it is consistent herewith.

Because no plasma biomarkers are associated with the response of aGVHD to therapy, a proteomic comparison was undertaken of plasma taken a median of two weeks after therapy initiation from 10 patients with complete response by day 28 post-therapy and 10 patients with progressive aGVHD on therapy. The lead biomarker, suppressor of tumorigenicity (ST2), was measured at aGVHD onset in plasma from 381 patients to determine the association of resistant aGVHD and 6-month mortality after treatment. Patients were then stratified for risk of non-relapse mortality (NRM) at initiation of therapy using the strongest clinical predictor, aGVHD grade, and the strongest biomarker, ST2. These preliminary data have shown that the biomarker value was more important than the clinical grade. ST2 is a recently discovered member of the interleukin-1 receptor family whose only known ligand is interleukin-33 (IL-33). Plasma IL-33 concentrations were similar in patients who had a response and those who did not (data not shown).

### Example 2

The ability of ST2 early in the transplant course was evaluated for its ability to predict the occurrence of aGVHD and 6-month NRM as described in US Publication No. 2013/0115232, published May 9, 2013, which is incorporated herein by reference to the extent it is consistent herewith.

ST2 concentrations were measured at D0, D14, and D21 post-transplant in three cohorts of 296, 302 and 75 patients who did not develop GVHD before D35. ST2 concentrations at D14 correlated with 6-month NRM. In multivariate analysis including the clinical characteristics of conditioning intensity, age, disease status, donor source, and HLA-match, patients with high ST2 concentrations at D14 also had increased risk of 6-month NRM after adjustment for the clinical characteristics indicating that when measured as early as D14 post-transplant, ST2 was a better predictor of mortality than the other known risk factors.

There are two ST2 isoforms: a membrane-bound form expressed on hematopoietic cells, particularly type 2 and 9 helper T (Th2, Th9) cells, which play a role in Th2- and Th9-mediated diseases such as asthma, and a soluble form, secreted by endothelial cells, epithelial cells, and fibroblasts in response to inflammatory stimuli. Soluble ST2 acts as a decoy receptor for IL-33 and drives Th2/Th9 cells toward a Th1-cell phenotype, which may be important in the pathophysiology of GVHD.

The ST2 assay was validated for its analytical performance in the Viracor-IBT laboratories. ST2 ELISA assay performance has been established by the following parameters: 1) accuracy (i.e., closeness of the test result to the true value); 2) upper and lower limit of detection; 3) intra- and inter-assay variability; 4) reliability; and 5) reproducibility.

### Examples 3-9

### Materials and Methods

Patients and Samples

Heparinized peripheral blood mononuclear cells (PBMCs), peripheral blood and plasma samples were collected weekly during 4 weeks and then monthly after hematopoietic (HSCT) and at onset of clinical key events (symptoms of GVHD, e.g., diarrhea), under protocols approved by the University of Michigan institutional review board and after proof of informed consent. PBMCs were isolated from peripheral blood by density gradient separation and were stored frozen at −120° C. in liquid nitrogen. Plasma samples were stored at −80° C.

All patients received GVHD prophylaxis with at least two agents, including a calcineurin inhibitor (>90% received tacrolimus). All patients received T-replete graph and GVHD prophylaxis. Samples were not analyzed if methylprednisolone at a dose higher than 1 mg/kg was administered 48 hours or more before sample collection. Clinical data abstraction was aided with use of the electronic medical record search engine (EMERSE).

Intact Protein Analysis System (IPAS)

An unbiased top-down proteomic approach based on high-resolution mass spectrometry (Intact Protein Analysis System (IPAS)) was used to identify candidate biomarkers followed by high-throughput sandwich ELISA to validate the candidate proteins. IPAS was performed on plasma samples taken 14 days before clinical manifestations of GI GVHD. Plasma biomarkers were selected that were increased at least 1.5-fold in plasma from GI GVHD patients compared to patients without GVHD at matched time points.

Multicolor Flow Cytometry

Frozen PBMCs phenotyping of cell surface markers was performed using CD4, CD25, CD127, CD146 (clone P1H12), CCR5 (clone 2D7), ICOS (CD278), CD45RA, and CCR7. Intracellular staining of transcription factors was performed after fixation and permeabilization provided by the manufacturer's recommendations using the buffer set (eBioscience). CD4 T conventional cells (T convs) were defined as CD4⁺CD25^{lo}CD127⁺. Regulatory T cells (T regs) were defined as CD4⁺CD25⁺CD127⁻FOXP3⁺. Fluorescence-activated cell sorting (FACS) analysis was performed using an 8 color-Canto II flow cytometer (BD Bioscience) and FlowJo software for Mac (TreeStar Inc., OR). Absolute counts of T cell subset were calculated by multiplying the frequency of T cells on lymphocytes by the absolute lymphocyte count (ALC) obtained by automated method. The values represented in bar graphs as mean±standard error of the mean (SEM). For intracellular cytokine staining, cells were stimulated with PMA (50 ng/ml) (Sigma) and Ionomycin (1 μg/ml) (Sigma) and Brefeldin A (3 μg/ml) (eBioscience) for 5 hours in a 37° C. and 5% CO₂ incubator. Intracellular cytokines and transcriptional factors were stained with FoxP3 staining Kit (eBloscience). Stained cells were analyzed with Attune (Invitrogen) Flow Cytometer and FlowJo software.

Immunohistochemistry

Biopsies from GI GVHD and non-GVHD enteritis patients were obtained and performed per institutional guidelines. GVHD was confirmed by duodenal or colonic biopsy in 61 of 71 GI GVHD patients. Biopsies were graded as discussed in Lerner et al., Histopathology of graft-vs.-host reaction (GvHR) in human recipients of marrow from HL-A-matched sibling donors. Transplantation proceedings 6, 367 (December, 1974). CD146 monocolonal antibody, clone sc-374556, was purchased from Santa Cruz Biotechnology Immunostaining was performed by the tissue core of the University of Michigan Comprehenisive Cancer Center for a subset of 18 GI GVHD and 10 non-GVHD enteritis patients post-HCT. All stained GI sections were coded, and two pathologists counted positive cells or positive blood vessels in a blind manner at 400× magnification per 10× optical field.

TCRβ Regions Sequencing of T Cell Populations

The ImmunoSEQ assay (Adaptive Biotechnologies, Seattle, Wash.) employs primers to 60Vβ and 13Jβ segments and utilizes multiplex PCR to amplify the rearranged CDR3 regions of the T cell receptor, spanning the variable region formed by the junction of V, D, and J segments and any non-templated insertions. Number and frequency of resulting nucleotide sequences describes the diversity of the T cell repertoire as measured by entropy/Shannon's diversity index. Higher entropy scores reflect greater diversity within a T cell population. This approach directly determines number of unique TCR sequences, frequency of individual sequences and degree of expansion or maintenance of specific T cell clones between samples. To profile TCRβ repertoires of CD146CCR5 double positive and double negative CD4 T cells, the two populations were sorted from PB cells prepared by Ficoll (GE Healthcare, Pittsburgh, Pa.) density gradient centrifugation from PB leukopacks from healthy donors purchased from the Central Indiana Blood Center under an institutional review board-approved protocol. Before sorting the double positive CD4 T cells, CD146⁺ cells were enriched with human CD146 (Miltenyi). PB cells and enriched CD146⁺ cells were then stained with antibodies to CD3, CD4, CD25, CD127, CD146, and CCR5 (eBioscience). The double positive and double negative CD4 T cells were sorted with a BD SORP Aria on the gated CD4⁺CD25^{lo}CD127⁺ population in the CD146⁺ enriched cells and total PB cells, individually. Sorted CD146⁺CCR5⁺ (120K) and CD146⁻CCR5⁻ (250K) Tconvs were frozen and sent to Adaptive Biotechnologies for DNA extraction and high-throughput TCRβ sequencing with the ImmunoSEQ platform. Clonality, which is a measure equal to the inverse of the normalized Shannon entropy of all productive clones in a sample, can be compared directly between samples of varying sizes. Therefore, clonality of the TCRβ sequences was compared between the two populations. Values for clonality ranged from 0 to 1. Values close to 1 represented samples with one or a few predominant clones dominating the observed repertoire and values close to 0 represented more polyclonal samples.

Sort and Nanostring

Human PB leukopacks from anonymous healthy donors were obtained from Indiana Blood Center under an Institutional Review Board approved protocol. Peripheral blood mononuclear cells (PBMCs) were isolated within 24 hours after blood draw by Ficoll (GE Healthcare, Pittsburgh, Pa.) density gradient centrifugation according to the manufacturer's instructions, then PBMCs were resuspended in FACS buffer (phosphate-buffered saline (PBS) supplemented with 2% fetal bovine serum (FBS)) at a density of 10×10⁶ cells per milliliter.

For cell staining, all antibodies were purchased from eBioscience unless stated otherwise. To prevent aspecific binding of the antibodies, the cells were pre-incubated with human Fc receptor binding inhibitor (eBioscience, San Diego, Calif.) for 10-20 minutes at 4° C., subsequently incubated for 30 minutes at 4° C. with R-phycoerythrin (PE)-conjugated anti-CD146, fluorescein isothiocyanate (FITC)-conjugated anti-CD4, allophycocyanin (APC)-conjugated anti-CD25, PE-Cyanine7(PE-Cy7)-conjugated anti-CCR5, APC-EFLUOR0780-conjugated anti-CD127 (eBioscience). Thereafter, the stained cells were washed twice with FACS buffer, and resuspended in PBS with 0.5% FBS for cell sorting using an iCyt Reflection (Sony Biotechnology Inc.). Cell preparations with the corresponding PE, PE-Cy7, APC or APC-EFLUOR®780 conjugated isotype-matched control monoclonal antibodies were also prepared. Cell sorting was performed on iCyt Reflection (Sony Biotechnology Inc.). Four populations (CD146⁺CCR5⁺ Tregs, Treg excluding CD146⁺CCR5⁺, CD146⁺CCR5⁺ Tconvs, Tconvs excluding CD146⁺CCR5⁺) were sorted. Then, sorted cells were direct lysis by RTL buffer (QIAGEN GmbH) on ice. The cell concentration for lysis was 2,000-10 k cells per microliter with a total of 5 μl by RTL buffer. Lysis samples were frozen in liquid nitrogen immediately then stored at −80° C. or on dry ice for Nanostring analysis.

For Nanostring analysis, all reporter and capture probes were obtained from NanoString Technologies (Seattle, Wash.) to be compatible with its NCOUNTER® Analysis System. The nCounter GX Human Immunology Kit, which includes more than 500 clinically relevant immune genes, was used. Manufacturer's instructions of NanoString for hybridization and detection from the lysis sample were followed.

T Cell Polarization

T cells were purified from PB cells prepared from fresh leukopacks (Central Indiana Blood Bank) as described above. Naïve CD4+ T cells were negatively isolated using a naïve CD4+ T cell isolation kit (Miltenyi Biotec, Germany). Total CD4⁺ T cells were negatively or positively isolated using a CD4⁺ T isolation kit (Miltenyi Biotec). Tconvs were positively selected with CD4 microbeads after depletion of CD25 cells with CD25 microbeads (Miltenyi). CD146⁺CD4⁺ T cells and CD146⁻CD4⁺ T cells were isolated from total CD4⁺ T cells using CD146 microbeads (Miltenyi Biotec). CD8 T cells were positively selected using CD8 microbeads (Miltenyi). Purification of isolation was >95%.

For T cell activation, 0.5×10⁶ T cells were plated in a 48-well flat bottom plate with CD3/CD28 or CD3/ICOS coated M-450 Tosylactivated Dynabeads (Invitrogen, Carlsbad, Calif.). The bead-to-cell ratio was 1:5. Cells were cultivated in T cell expansion medium (Invitrogen) in a 37° C. and 5% CO₂ incubator.

For Th1 polarization, IL-2 (2 ng/ml), IL-12 (10 ng/ml) (R&D system), and neutralizing antibodies against IL-4 (10 μg/ml) (eBioscience, Inc., San Diego, Calif.) were added on day 0 (D0). For Th17 polarization, IL1β (20 ng/ml), IL-6 (30 ng/ml), IL-23 (30 ng/ml), TGFβ (2 ng/ml) (R&D Systems, Minneapolis, Minn.) and neutralizing antibodies against IL-4 (5 μg/ml) and IFNg (2 μg/ml) (eBioscience) were added on D0. The polarizing cytokines and antibodies were maintained throughout the 7-day cell culture period.

In Vitro Stimulation of Tregs

CD4+CD25+CD127− T cells were purified from PB cells prepared from fresh leukopacks (Central Indiana Blood Bank) using the CD4+CD25+CD127− Regulatory T Cell Isolation Kit II (Miltenyi). For stimulation of Tregs, 0.1×10⁶ T cells were plated in 96-well round bottom plates and stimulated with anti-CD3/anti-CD28 or anti-CD3/anti-ICOS antibody-coated Dynabeads in the presence of 500 U/mL IL-2 for 8-9 days.

Mixed Lymphocyte Reactions (MLRs)

PB cells were prepared from healthy donors (Central Indiana Blood Bank). In an allogeneic mixed lymphocyte reaction, PB cells from one donor were irradiated at 3000 cGy, used as the stimulator, and mixed in a 1:1 ratio with PB cells or purified CD4 T cells from another donor, used as the responder. For autologous MLRs, irradiated PB cells were mixed with non-irradiated PB cells from the same donor. Cells were cultured in T cell expansion medium at 37° C. in 5% CO₂ for 8 days before analysis.

Flow Cytometry for In Vitro Assays

Differentiated T cells or MLR cells were analyzed by flow cytometry for cell surface expression of CD4, CD146, CCR5 (BD Biosciences), CD161, CXCR6, and IL-23R. All antibodies were obtained from eBioscience unless otherwise indicated. For intracellular cytokine staining of IFN-γ and IL-17, cells were stimulated with PMA (50 ng/ml; Sigma-Aldrich) and ionomycin (1 μg/ml; Sigma-Aldrich), and brefeldin A (3 μg/ml) (eBioscience) for 5 hours at 37° C. in 5% CO₂. Intracellular cytokines were stained with the intracellular fixation and permeabilization buffer set (eBioscience). Stained cells were analyzed using an Attune Flow Cytometer (Life Technologies) and FlowJo software.

Lentiriral shRNA-Mediated Knockdown of CD146 on CD4 T Cells

The pCL2EGw.THPC lentiviral vector used to generate CD146 shRNA and control shRNA contains a viral spleen focus forming (SFFV) promoter and a histone 1 promoter driving enhanced green fluorescence protein (EGFP) and shRNA expression, respectively. To generate shRNA directed against CD146, the following three target sequences of the human CD146 gene were inserted between the Mlu I and Cla I sites of pCL2EGw.THPC: shRNA1 5′-GGAACTACTGGTGAACTAT-3′ (SEQ ID NO:1), shRNA2 5′-AGAGCGAACTTGTAGTTGA-3′ (SEQ ID NO:2), and shRNA3 5′-CCAACGACCTGGGCAAAAA-3′ (SEQ ID NO:3), which was used as a negative control shRNA because shRNA3 had no effect on CD146 expression on CD4 T cells.

To produce lentiviral particles, the CD146 shRNA or the control shRNA vector was co-transfected with the pCD/NL-BH helper plasmid and the pczVSV-G envelope plasmid into human embryonic kidney cells (HEK293T, ATCC, CRL-3216) as described previously in Wiek et al., The Biochemical Journal 465, pp. 103-114 (2015), published online EpubJan 1 (10.1042/BJ20140813). Viral supernatants were harvested 48 hours after transfection, filtered through a 0.45-μm filter, and concentrated by high speed centrifugation at 10,000 rpm for 2 hours. To knockdown CD146 expression on Th17-differentiated T cells with CD146 shRNA1, naïve CD4 T cells were stimulated with anti-CD3/CD28 beads under Th17 conditions for 24 hours and transduced with CD146 and control shRNA lentivirus immobilized on Retronectin-coated 24-well plates. After 6 days post-infection, the Th17 cells were re-stimulated with anti-CD3/CD28 beads under Th17 conditions for 6 more days to achieve maximal knockdown. To knockdown CD146 expression on Th17-differentiated T cells with CD146 shRNA2, naïve CD4 T cells were stimulated with anti-CD3/ICOS beads under Th17 conditions for 24 hours, transduced with the CD146 and control shRNA lentivirus, and cultured for 5 additional days. Transduced cells were then sorted based on GFP expression and used for in vitro assays. For in vivo functional analysis of lymphocytic CD146, CD4 T cells were pre-activated with anti-CD3/CD28 beads and IL-2 (20 U/ml) for 24 hours, infected with CD146 and control shRNA lentivirus as described above, and expanded in the presence of IL-2 for 4-6 days. Then GFP-expressing cells were sorted.

siRNA-mediated CD146 knockdown in human umbilical vein endothelial cells (HUVECs)

The Silencer Select siRNA targeting the human CD146 gene (s8573, sense strand 5′-GGAACUACUGGUGAACUAUtt-3′ (SEQ ID NO:4) and antisense strand 5′-AUAGUUCACCAGUAGUUCCtg-3′ (SEQ ID NO:5)) and the Silencer Select Negative Control No. 1 siRNA were obtained from Life Technologies and transfected into HUVECs using the TransIT-TKO Transfection Reagent from Mirus Bio according to the manufacturer's instructions. HUVEC cell line was from ATCC (PCS-100-010) and was tested for absence of mycoplasma contamination. Four days after transfection, significant reduction of CD146 was achieved, and the cells were used for the transmigration experiments.

Transmigration Assay

The transmigration of T cells through human microvascular endothelial cells (HMVECs) (Lonza Biosciences, CC-2543) was assayed using transwells with a 3-μm pore size (Costar) and HMVECs between passages 7 and 10. HMVECs (2×10⁴) were grown on 50 μg/ml collagen-coated transwell inserts in EGM-2MV medium (Lonza Biosciences) for 3 days, followed by treatment with fresh medium containing tumor necrosis factor (TNF)-α (10 ng/ml, Sigma-Aldrich) for 24 hours. The HMVECs were tested for absence of mycoplasma contamination. FACS-sorted CD146⁺ and CD146⁻ cells from fresh CD4 T cells among healthy donor PB cells, Th1 cells, or Th17 cells (1×10⁵) at day 6 of differentiation, or sorted GFP⁺ lentivirally transduced Th17 cells were added to the top chamber and left to transmigrate through the HMVEC monolayers for 24 hours in 1:1 mixture of EGM-2MV and T-cell growth media. T cells that migrated to the lower chamber were then collected and counted. The percent transmigration was calculated as the ratio of the number of transmigrated T cells to the number of input T cells. Transmigration experiments were performed in duplicate.

Chemotaxis Assay

MLR cultures with purified CD4 T cells as responders were stained with antibodies against CD146 and CCR5 (Clone REA245, Miltenyi) and sorted for CD146CCR5 double-positive CD4 T cells. After culture in T-cell growth medium supplemented with 4 U/ml IL-2 overnight, the cells were washed and pre-incubated with the CCR5 antagonist Maraviroc (10 μM, Selleckchem), or DMSO control for 30 minutes at 37° C. in chemotaxis medium (RPMI-1640 and 0.5% BSA). Approximately 0.5-1×10⁵ cells were added to the top chamber and 600 μl of chemotaxis medium with or without 100 nM CCL14 (Met-(Gly28-Asn93), Biolegend) or a mixture of CCL14 and 50 nM CCL5 (Biolegend) was added to the bottom chamber of a 24-well Transwell system (polycarbonate filter with 3-μm pore size, Costar). After incubation for 6 hours at 37° C., cells from the lower compartment were collected and counted by flow cytometry (Attune, Life Technologies). Chemotaxis is represented as the ratio between the number of T cells migrating towards chemokines and the number of cells that underwent spontaneous migration.

CD146 Knockout Donor T Cells in Murine Allogeneic Acute GVHD Models

T cells from the CD146 knockout (KO) mice and wild-type (WT) controls (8-20 weeks of age; female and male donors) on the H-2^{b} background, as described in Wu et al., The American journal of pathology 182, pp. 819-827 (2013), published online EpubMar (10.1016/j.ajpath.2012.11.005), were used to induce GVHD in two established murine models as described in Tawara et al., Clinical cancer research: an official journal of the American Association for Cancer Research 17, pp. 77-88 (2011), published online EpubJan 1 (1078-0432.CCR-10-1198 [pii] 10.1158/1078-0432.CCR-10-1198)). Briefly, Balb/c (H-2^{d}, MHC-mismatched) and C3H.SW (H-2^{b}, minor histocompatibility antigen-mismatched) (8-12 weeks of age; female and male) recipient mice received 900 and 1100 cGy total body irradiation, respectively, on day-1 (D-1). Recipient mice were injected intravenously (IV) with T-cell depleted (TCD) bone marrow cells (5×10⁶) from WT mice together with either WT or KO splenic T cells (1×10⁶ WT or CD146^{-/-} B6 T cells for Balb/c and 2×10⁶ for C3H.SW) on day 0 (D0). Donor T cells were purified using the murine Pan T Cell Isolation Kit and TCD bone marrow cells were prepared with CD90.2 Microbeads (Miltenyi). Mice were housed in sterilized microisolator cages and maintained on acidified water (pH<3) for 3 weeks as described in Reddy et al., The Journal of clinical investigation 118, pp. 2562-2573 (2008), published online EpubJul (10.1172/JCI34712)). Mice were monitored for survival daily, assessed for clinical GVHD scores weekly as described in Cooke et al., Blood 88, pp. 3230-3239 (1996), published online EpupOct 15, and euthanized when the clinical scores reached 6 according to animal protocols. Blinding and randomization was not used.

Human-to-Mouse Xenogeneic Model of GVHD with CD146 or Control shRNA Knockdown Human T Cells

Immunodeficient NOD/scid/IL-2Rγ^{-/-} (NSG) mice (8-14 weeks of age; female recipients) were obtained from the In Vivo Therapeutics Core at the Indiana University Simon Cancer Center, housed under specific pathogen-free conditions, and maintained on food supplemented with Uniprim and acidic water. All procedures were performed in compliance with protocols approved by the institutional animal care and use committee and institutional biosafety committee. Twenty-four hours after total body irradiation at 300 or 350 cGy (for survival), NSG mice were injected IV with 1-2×10⁶ sorted human CD4 T cells lentivirally transduced with the CD146 or control shRNA vector. In some experiments, maraviroc dissolved in DMSO at a concentration of 100 mg/ml or an equal volume of DMSO alone (vehicle) was diluted in 200 μl PBS and injected into mice intraperitoneally (10 mg/kg) once daily from the day before T-cell transplantation and until 21 days afterwards. Mice were monitored daily for survival and scored twice a week for GVHD signs as described above.

Spleen and small and large intestines were harvested from transplanted NSG mice. Single cell suspension of spleens was prepared for cell analysis. To isolate single intestinal cells, the small and large intestines were flushed with PBS to remove fecal matter, cut open longitudinally, washed with PBS to remove mucus, cut into small pieces, suspended in 10 ml Dulbecco's Modified Eagle's Medium (DMEM; Life Technologies) with 4% bovine serum albumin, 200 μg/ml collagenase B, and 10 U/ml DNase I (Roche Diagnostics) at 37° C. and 250 rpm for 1.5 hours. Digested mixtures were then diluted with 30 ml DMEM, filtered through 70-μm strainers, and centrifuged for 10 minutes at 500 g. Cell pellets were suspended in 5 ml 80% Percoll, overlaid with 8 ml 40% Percoll, and spun at 800 g for 25 minutes at 4° C. without braking. Single suspended intestinal cells were collected from the interface. Cells were stained with Cell Viability Dye (eBioscience), and cell surface staining of murine CD45 and human CD4, CD146, and CCR5 or intracellular staining of IL-17 and IFN-γ after PMA and ionomycin stimulation were performed as described above. At day 45 post-transplantation, 37.9±2.9% and 11.0±3.7% (n=10) human CD4 T cells in the intestines expressed CD146 in the control shRNA group and CD146 shRNA group, respectively.

Histopathological Analysis of Xenogeneic GVHD

Between 26-38 days post transplantation, intestine, liver, skin, and lung were collected from NSG mice receiving transduced CD4 T cells and treated with maraviroc or vehicle control. Hematoxylin and eosin (H&E) stained slides of formalin-preserved tissues were prepared by the Pathology Department at Indiana University Medical Center. The slides were coded without reference to previous treatment and examined in a blinded fashion. A semi-quantitative scoring system was used to assess abnormalities known to be associated with GVHD (Hill et al., The Journal of clinical investigation 102, pp. 115-123 (1998)). After scoring, the codes were broken and the data were compiled.

Statistical Analysis

Differences in characteristics between patient groups were assessed using Kruskal-Wallis test for continuous values and χ² tests of association for categorical values. Frequencies of T cells subsets were compared using an unpaired t-test (Student's t-test). Differences in IHC staining of GI biopsies were calculated using Fisher's exact test. Receivers operating characteristic (ROC) and area under the curves (AUC) were estimated non-parametrically. The squared Pearson correlation coefficient was used for correlation analysis between markers. Differences in immunohistochemical staining of GI biopsies were calculated using unpaired t-tests. NRM and relapse mortality were modeled with cumulative incidence regression methods as described by Fine and Gray, J Am Stat Assocn, 94, pp. 496-509 (1999). OS was modeled with Cox regression methods. In vitro data were analyzed by using unpaired t-tests or paired t-tests for in vitro transmigration assays performed with the same donor or one-way analysis of variance (ANOVA). P-values<0.05 were considered to be statistically significant.

### Example 3

In this Example, GI-specific biomarkers for GVHD were determined.

Previously, systemic, skin- and GI-specific plasma biomarkers present at clinical GVHD onset have been identified, and more recently biomarkers for treatment responsiveness (see Examples 1 and 2). However, in order to identify early GI-specific biomarkers prior to GVHD onset, proteomics on a pool of 10 plasma samples taken 14 days prior to clinical manifestation of GI GVHD (labeled with a heavy isotope) was performed. A pool of 10 matched controls (labeled with a light isotope) was also analyzed. The isotopes allowed for comparison of relative concentrations of proteins between the groups. The two pools were then subjected to tandem mass sprectrometry. Patient characteristics for the discovery phase are shown in Table 1. Candidate biomarkers that increased at least 1.5 fold in plasma from GI GVHD patients compared to HSCT patients without GVHD at matched time points were selected.

TABLE 1
Study patients characteristics

| | | | Non-GVHD | Skin | Skin | |
|---|---|---|---|---|---|---|
| | GI GVHD | No GVHD | Enteritis | GVHD 1^{st} | GVHD Only | |
| Total, n = 214 | n = 71 | n = 48 | n = 33 | N = 22 | N = 40 | p-value |
| Median age | 47 (0-65) | 48 (2-67) | 42 (3-65) | 47 (7-66) | 52 (1-65) | 0.44 |

Disease, %

| | | | | | | |
|---|---|---|---|---|---|---|
| Malignant | 96 (n = 68) | 92 (n = 44) | 91 (n = 30) | 96 (n = 21) | 93 (n = 37) | 0.84 |
| Other | 4 (n = 3) | 8 (n = 4) | 9 (n = 3) | 4 (n = 1) | 7 (n = 3) | |

Disease status at transplantation, %

| | | | | | | |
|---|---|---|---|---|---|---|
| Other/low/intermediate risk | 58 (n = 41) | 69 (n = 33) | 57 (n = 19) | 54 (n = 12) | 72 (n = 29) | 0.39 |
| High risk | 42 (n = 30) | 31 (n = 15) | 43 (n = 14) | 46 (n = 10) | 28 (n = 19) | |

Donor type, %

| | | | | | | |
|---|---|---|---|---|---|---|
| Related donor | 44 (n = 31) | 67 (n = 32) | 57 (n = 19) | 32 (n = 7) | 45 (n = 18) | 0.03 |
| Unrelated donor | 56 (n = 40) | 33 (n = 16) | 43 (n = 14) | 68 (n = 15) | 55 (n = 22) | |

Donor match, %

| | | | | | | |
|---|---|---|---|---|---|---|
| Matched donor | 75 (n = 53) | 87 (n = 42) | 88 (n = 29) | 73 (n = 16) | 85 (n = 34) | 0.17 |
| Mismatched donor | 25 (n = 18) | 13 (n = 6) | 12 (n = 4) | 27 (n = 6) | 25 (n = 6) | |

Conditioning regimen Intensity, %

| | | | | | | |
|---|---|---|---|---|---|---|
| High intensity | 62 (n = 44) | 73 (n = 35) | 70 (n = 23) | 64 (n = 14) | 62 (n = 25) | 0.73 |
| Intermediate/Reduced intensity | 38 (n = 27) | 27 (n = 13) | 30 (n = 10) | 36 (n = 8) | 38 (n = 15) | |
| Median after HSCT (range) Stage of | 29 (11-92) | 30 (14-131) | 28 (13-78) | 29 (10-85) | 28 (7-100) | 0.54 |
| GVHD at onset, % | | | | | | |
| 0 | 0 (n = 0) | 100 (n = 48) | 100 (n = 33) | 0 (n = 0) | 0 (n = 0) | n/a |
| I | 0 (n = 0) | 0 (n = 0) | 0 (n = 0) | 50 (n = 11) | 75 (n = 30) | |
| Skin stage 1 | 0 (n = 0) | 0 (n = 0) | 0 (n = 0) | 27 (n = 6) | 45 (n = 18) | |
| Skin stage 2 | 0 (n = 0) | 0 (n = 0) | 0 (n = 0) | 23 (n = 5) | 30 (n = 12) | |
| II | 65 (n = 46) | 0 (n = 0) | 0 (n = 0) | 45 (n = 10) | 25 (n = 10) | |
| Isolated skin stage 3 | 0 (n = 0) | 0 (n = 0) | 0 (n = 0) | 45 (n = 10) | 25 (n = 10) | |
| Isolated upper GI stage 1 | 24 (n = 17) | 0 (n = 0) | 0 (n = 0) | 0 (n = 0) | 0 (n = 0) | |
| Lower GI stage 1 | 41 (n = 29) | 0 (n = 0) | 0 (n = 0) | 0 (n = 0) | 0 (n = 0) | |
| III-IV | 35 (n = 25) | 0 (n = 0) | 0 (n = 0) | 0 (n = 0) | 0 (n = 0) | |
| Isolated skin stage 4 | 0 (n = 0) | 0 (n = 0) | 0 (n = 0) | 0 (n = 0) | 0 (n = 0) | |
| GI stage 1 | 1 (n = 1) | 0 (n = 0) | 0 (n = 0) | 0 (n = 0) | 0 (n = 0) | |
| GI stage 2 | 10 (n = 7) | 0 (n = 0) | 0 (n = 0) | 0 (n = 0) | 0 (n = 0) | |
| GI stage 3 | 10 (n = 7) | 0 (n = 0) | 0 (n = 0) | 0 (n = 0) | 0 (n = 0) | |
| GI stage 4 | 14 (n = 10) | 0 (n = 0) | 0 (n = 0) | 0 (n = 0) | 0 (n = 0) | |

Two lead proteins were identified: CD146, a cell adhesion and trafficking molecule expressed on a subset of CD4+ T cells and endothelial cells, particularly during inflammation, and the chemokine (C-C motif) ligand 14 (CCL14) that binds to the chemokine receptor CCR5 on T cells. CCR5 has been suggested to be required for T cell migration into inflamed intestine in a murine model. Their expression profiles on peripheral blood (PB) cells from 214 HSCT patients (71 GI GVHD, 48 no GVHD, 33 non-GVHD enteritis, 22 skin first GVHD, 40 isolated skin GVHD) at the onset of symptoms at a median of 29 days post-HCT, or at similar time points from patients with non-GVHD enteritis or without GVHD, were analyzed. The causes of non-GVHD enteritis are detailed in Table 2.

TABLE 2
Causes of non-GVHD enteritis in the validation set
Non-GVHD lower GI enteritis +/− upper GI symptoms: n = 25

| | |
|---|---|
| C. difficile infection | 39% (n = 13) |
| Diarrhea w/ negative biopsy (no other etiology) | 21% (n = 7) |
| N/V and diarrhea w/ negative biopsies (no other etiology) | 12% (n = 4) |
| Ulcerative esophagitis and diarrhea (negative biopsies) | 3% (n = 1) |

Non-GVHD upper GI enteritis without diarrhea (all biopsy
negative): n = 8

| | |
|---|---|
| Nausea/vomiting | 15% (n = 5) |
| Anorexia | 3% (n = 1) |
| Chemical gastropathy | 3% (n = 1) |
| H. pylori gastritis | 3% (n − 1) |

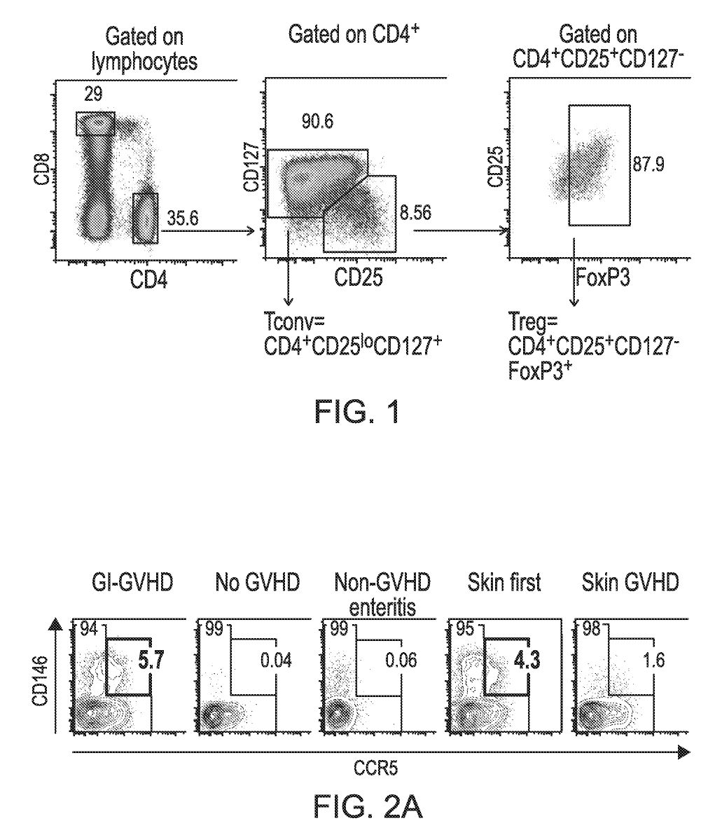

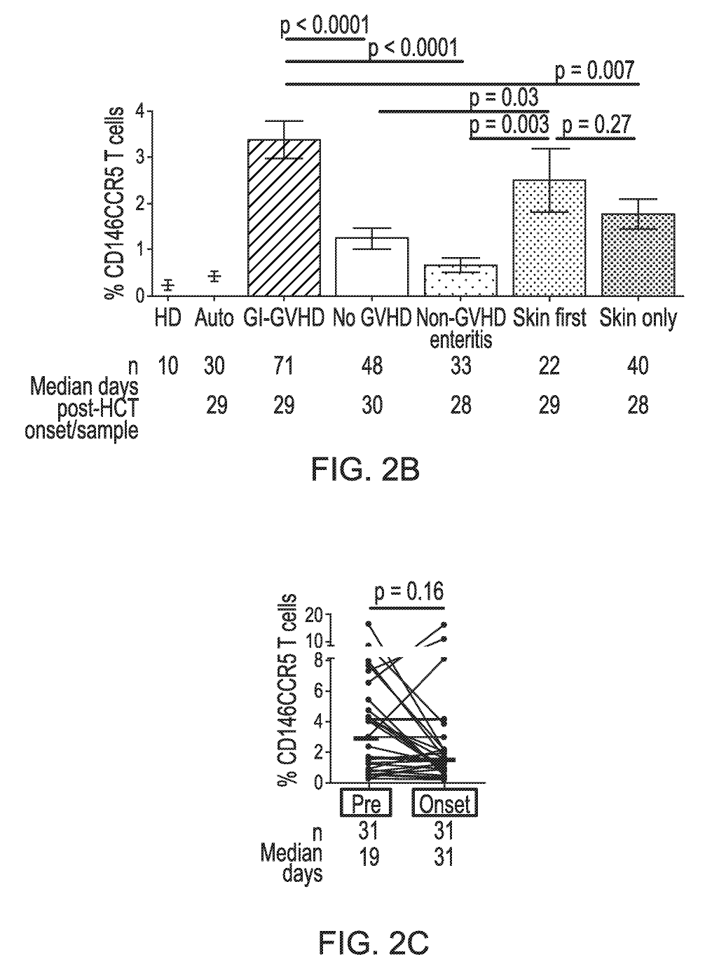

Conventional T cells were defined as CD4⁺CD25^{lo}CD127⁺ (see gating strategy in FIG. 1). The frequency of CD146+CCR5+T cells was significantly increased in GI GVHD patients compared to patients without GVHD, non-GVHD enteritis, or with isolated skin GVHD, as well as increased in patients who first experienced skin and then GI GVHD (FIGS. 2A & 2B).

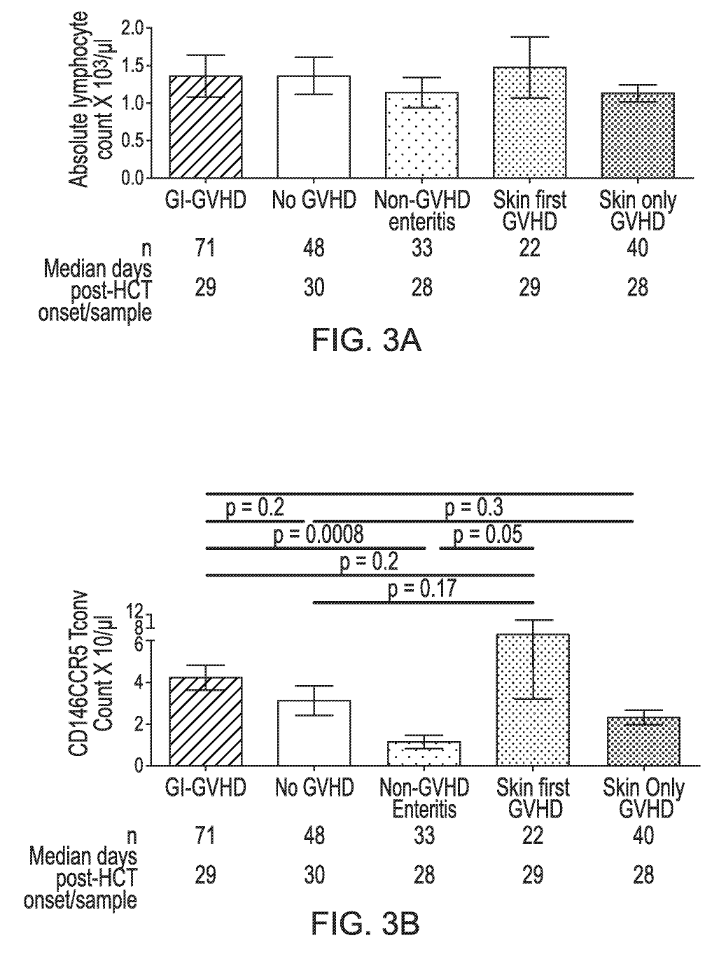

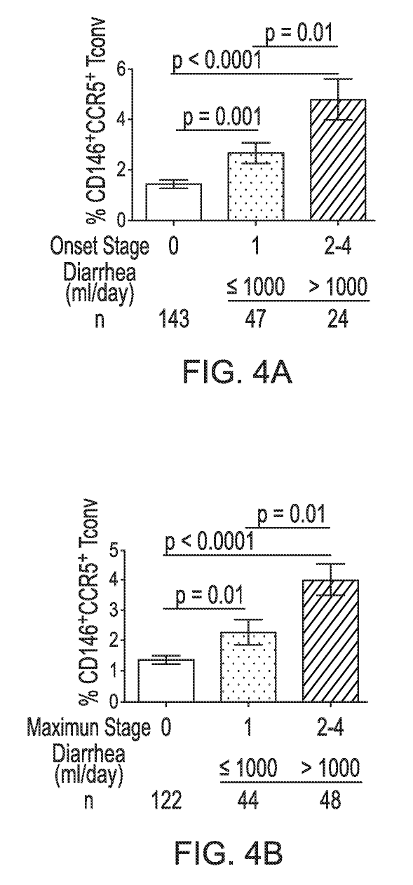

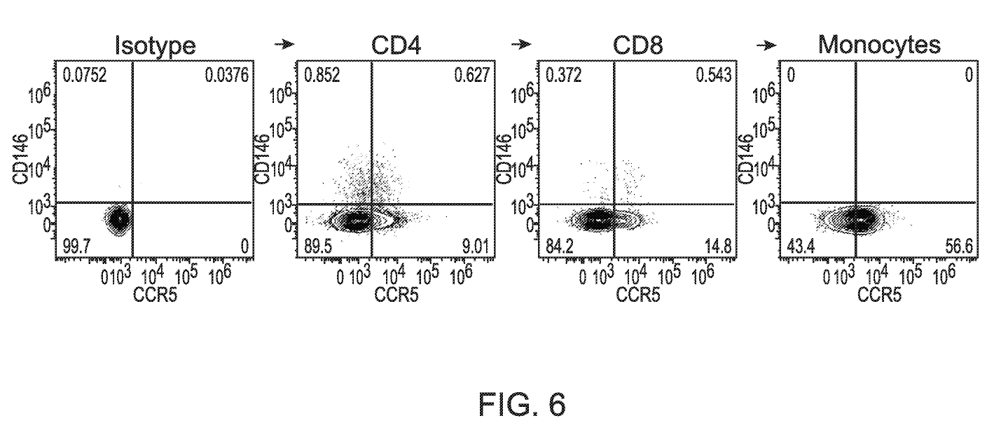

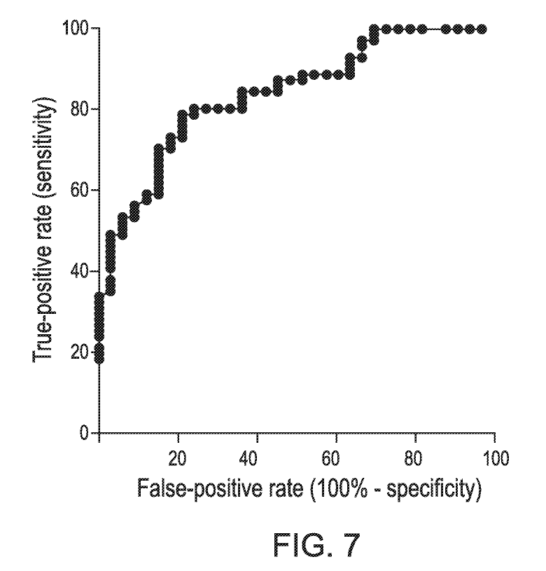

In addition, the absolute lymphocyte counts did not differ between groups, and the absolute counts of CD146CCR5 T cells in patients with GI GVHD or skin-first GVHD versus patients with non-GVHD enteritis remained significantly different (FIGS. 3A & 3B). The frequencies of T cells expressing only CD146 or CCR5 were not consistently different between the groups (FIGS. 4A & 4B), and CD8 T cells did not express CD146 in patients or healthy donors (healthy donors only shown at FIG. 6). The CD146CCR5 T cell frequency discriminated GI-GVHD from non-GVHD enteritis with an area under the receiver operating characteristic curve of 0.84 (see FIG. 7). These data suggest that the CD146CCR5 T cell frequency could be used as a diagnostic marker of GI-GVHD, particularly in comparison to non-GVHD enteritis.

### Example 4

In this example, the prognostic value of CD146CCR5 T-cell frequency was analyzed.

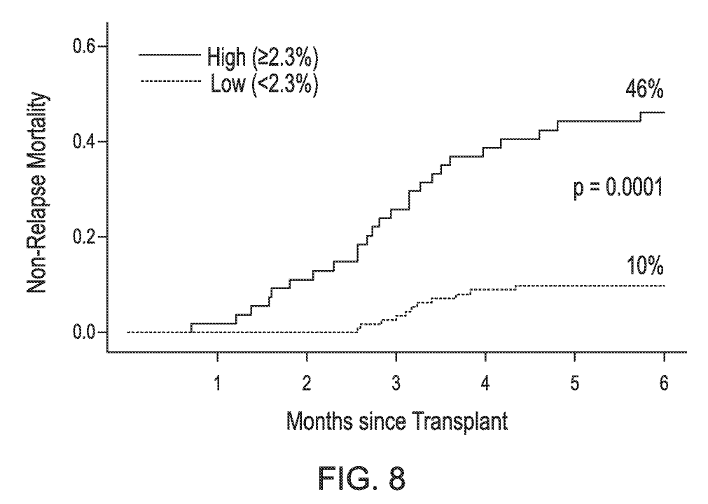

The strength of a biomarker is enhanced if it can be used to predict patient outcomes. The impact of the CD146CCR5 T-cell frequency on the 6-month non-relapse mortality in all patients with symptoms (GI-GVHD, non-GVHD enteritis, skin-only, and skin-first GVHD) was analyzed. It was found that the median CD146CCR5 T-cell frequency in patients with GI-GVHD (2.3%) could be used as a cutpoint for the risk of non-relapse mortality, with 46% of patients in the high-risk group experiencing non-relapse mortality compared to only 10% of patients in the low-risk group (p=0.0001, FIG. 8).

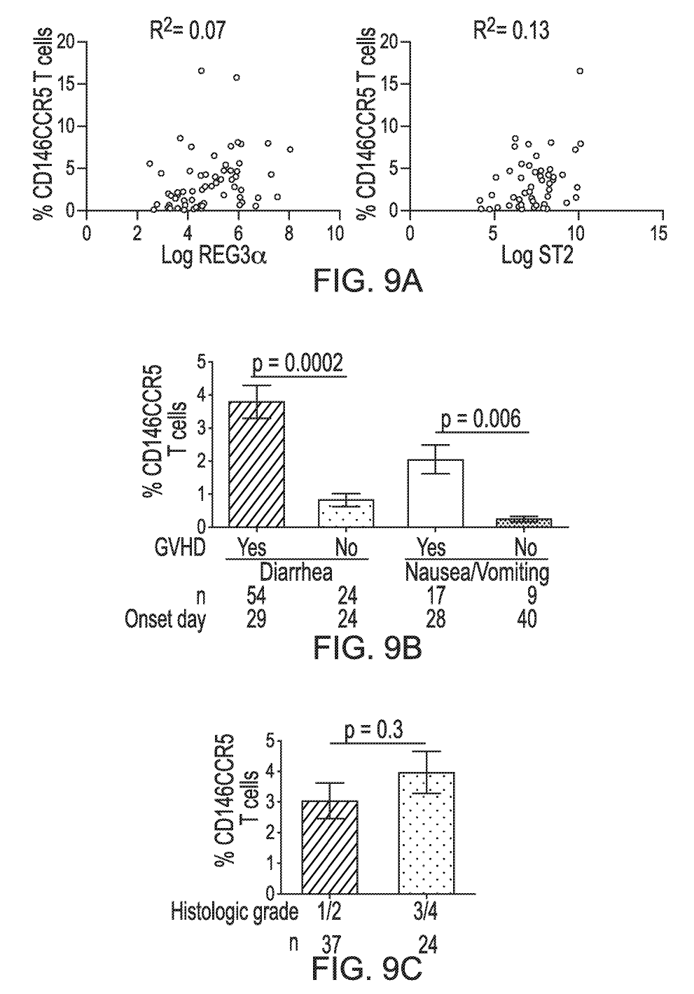

Further, a new biomarker improves diagnostic power only if it does not correlate with other known markers. Previous proteomics experiments revealed correlations between REG3α and ST2 with GI-GVHD (Ferrara et al., Blood 118, pp. 6702-6708 (2011); Wong et al., The New England journal of medicine 369, pp. 529-539 (2013))). It was found herein that these markers were not highly correlated with the CD146CCR5 T-cell population (FIG. 9A). The CD146CCR5 T-cell frequency in patients categorized according to the localization of GI symptoms was also analyzed: diarrhea vs. nausea/vomiting. The biomarker distinguished GI-GVHD from non-GVHD enteritis regardless of symptom localization, suggesting the CD146CCR5 T-cell subset appears early in the GVHD process, given that nausea/vomiting symptoms usually precede diarrhea symptoms (FIG. 9B). Furthermore, the CD146CCR5 T-cell frequency was not correlated with GI histologic severity, suggesting that these cells are not a product of mucosa damage, but rather systemic effectors (FIG. 9C).

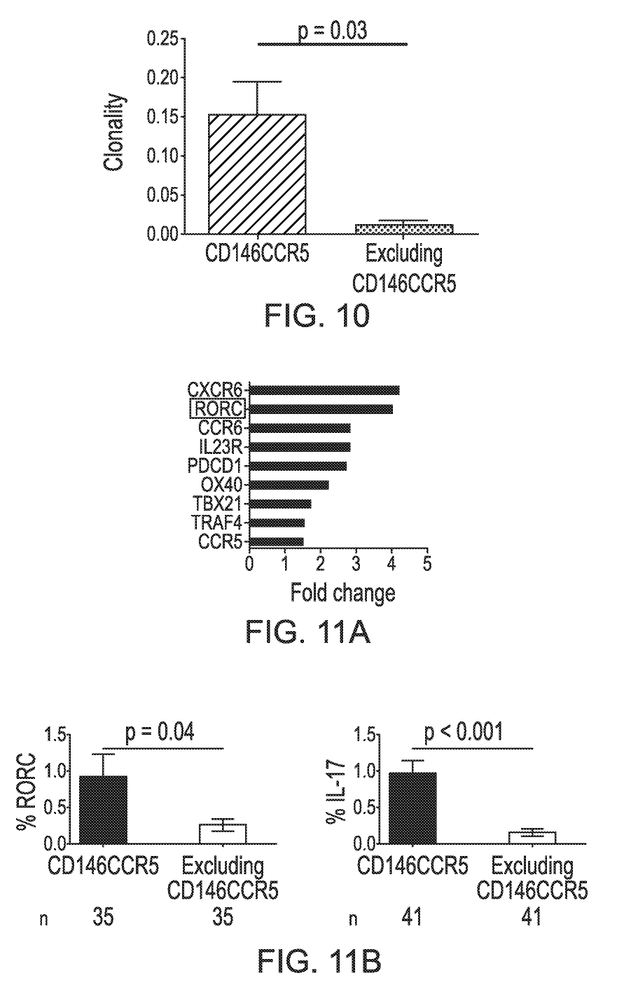

Based on these observations and the CD146 and CCL14 expression observed in samples prior to the onset of clinical signs, the prognostic value of CD146CCR5 T-cell frequency using 31 paired samples collected pre-GI-GVHD at a median of 19 days post-transplantation and at onset of GI-GVHD was then analyzed. The CD146CCR5 T-cell population was found to circulate in PB at a median interval of 14 days prior to the occurrence of GI-GVHD symptoms (FIG. 2C). Due to the early circulation of these cells after engraftment, their clonality was assessed by deep sequencing analysis of their T-cell repertoire first in total PB cells collected 14 days post-transplantation and before symptom onset from 5 GI-GVHD and 5 non-GVHD enteritis patients, and then on sorted CD146⁺CCR5⁺ and CD146⁻CCR5⁻ T-cell populations. Due to the limited quantities of material available, sorting of these cells was not possible from HCT recipients. Although the frequencies of the top 25 clones from the total PB cells did not differ between patients who later developed GI-GVHD or non-GVHD enteritis (FIG. 11), the sorted CD146⁺CCR5⁺ T cells from three healthy donors had significantly higher clonality than did the double-negative population (FIG. 10). These data suggest that quantitative assessment of the T-cell repertoire from circulating cells is more sensitive in enriched T-cell populations and requires advanced techniques to capture differences such as the one seen in the sorted T-cell populations.

### Example 5

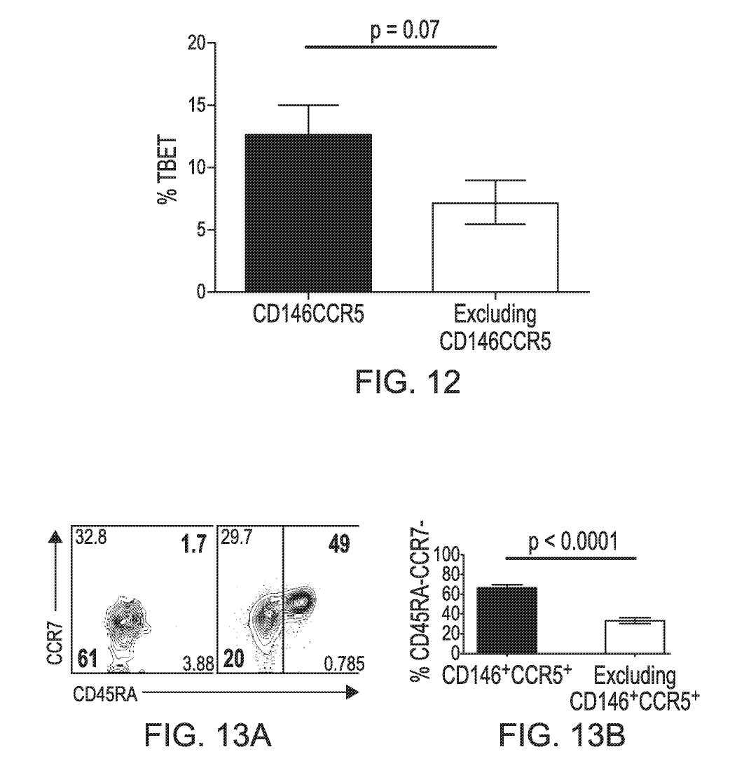

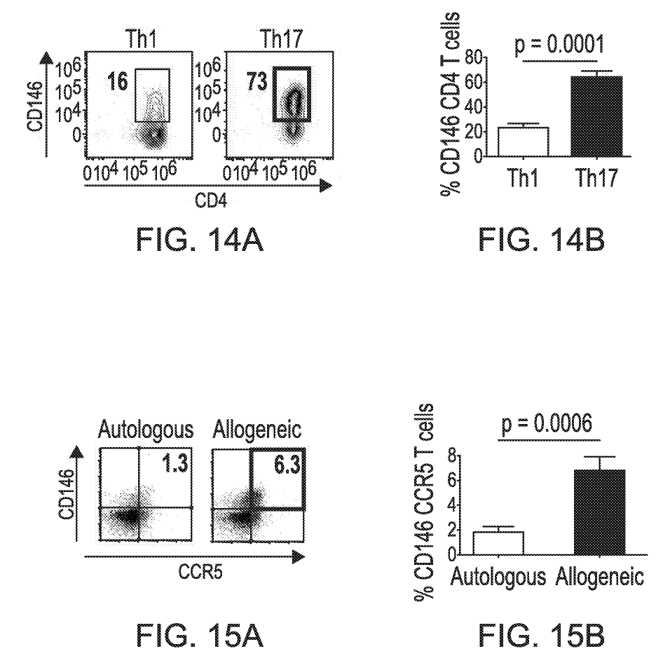

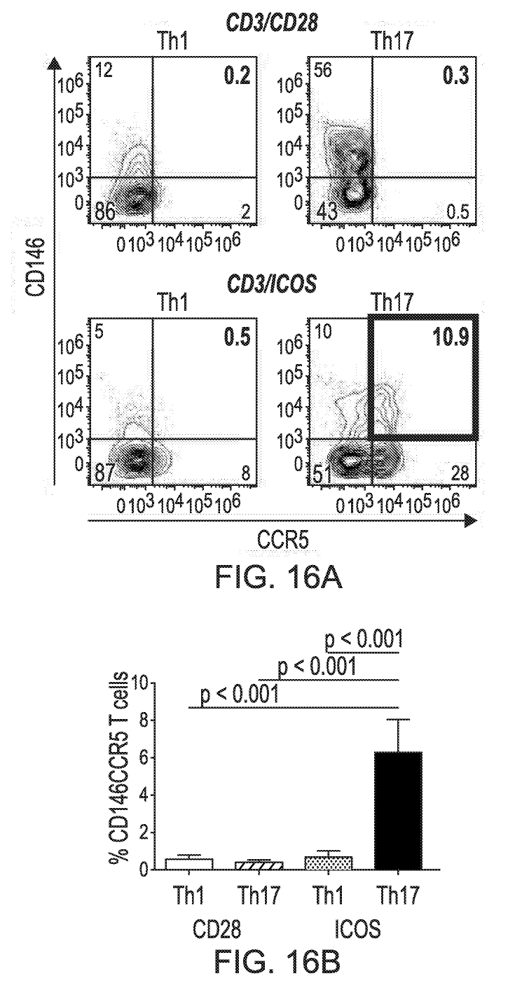

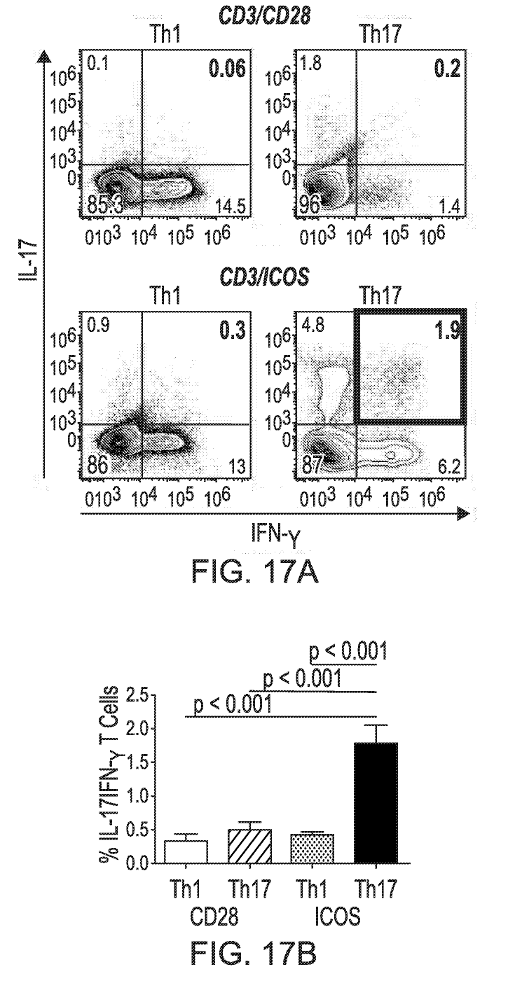

In this Example, the CD146CCR5 T-cell population was characterized and differential transcriptomes defined between sorted CD146CCR5 T cells and T cells excluding this population. Transcription of RAR-related orphan receptor C (RORC), a transcription factor essential for T helper (Th)17 development, was upregulated 4-fold, and this upregulation was confirmed at the protein level by intracellular staining of RORC and interleukin (IL)-17 in patients samples (FIGS. 11A & 11B). Intracellular staining for Th1-transcription factor TBET in the same patients' samples did not indicate significant upregulation (FIG. 12). The CD146CCR5 T-cell population is also characterized by an effector memory phenotype defined by the absence of CD45RA and CCR7 as compared to the T-cell population excluding the double positive population (FIGS. 13A & 13B). Consistent with data from patient samples, CD146 expression in in vitro Th17-differentiated cells was 3-fold greater than in Th1-differentiated cells (FIGS. 14A & 14B). It was also verified that CD146CCR5 T cells were induced upon allogeneic reactivity (FIGS. 15A & 15B). Inducible T-cell co-stimulator (ICOS) stimulation is critical for the development of human Th17 cells, and it was found that ICOS stimulation and Th17 differentiation conditioning induced the CD146CCR5 population (FIGS. 16A & 16B). This conditioning also increased the number of cells co-expressing IL-17/interferon (IFN)-γ (FIGS. 17A & 17B).

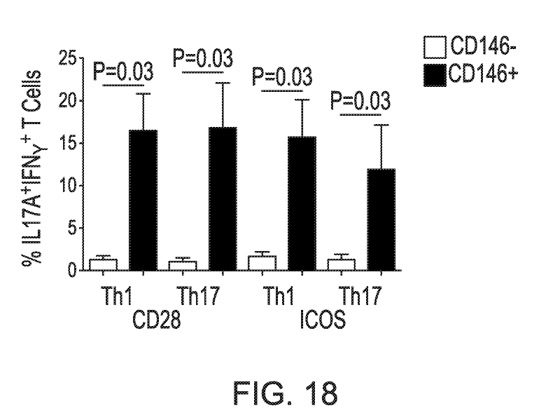

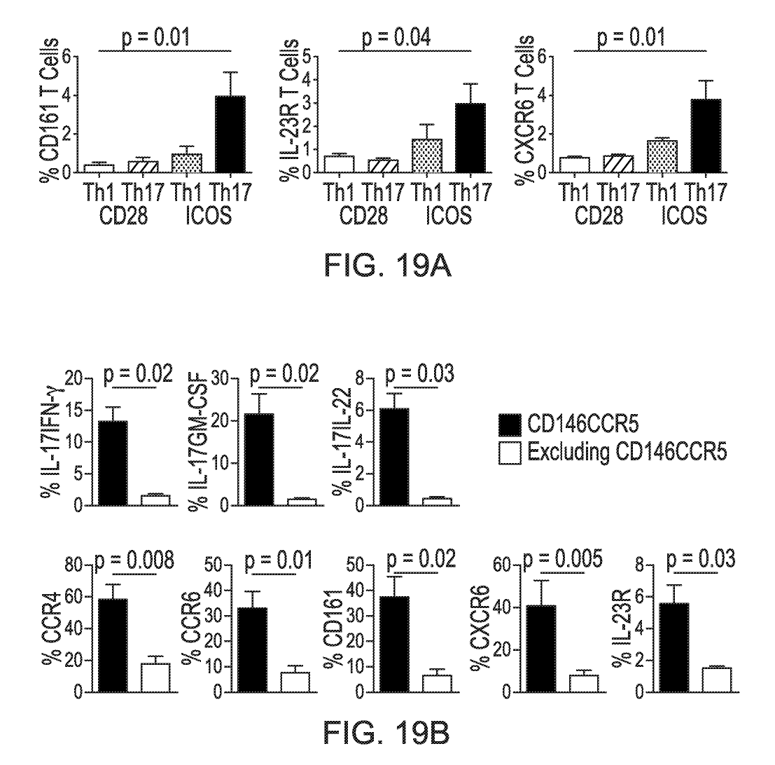

Additional experiments were performed and it was found that sorted mature CD146⁺ T cells expressed equivalent amounts of IL-17 after in vitro differentiation under Th1 or Th17 conditions with CD28 or ICOS costimulation (FIG. 18). These data suggest that the CD146CCR5 T cells are antigen-experienced and Th17-committed. Other Th17 markers such as CD161, IL-23R, and CXCR6 were all expressed at higher levels in T cells differentiated with Th17 conditioning and ICOS (FIG. 19A), and specifically, on the double-positive T cells as compared to the T cells excluding this population (FIG. 19B). Overall, these data show that the CD146CCR5 T-cell population is Th17-committed and increased by ICOS stimulation, linking these cells to two known driving forces for the induction and amplification of the GVHD effector phase, which results in direct and indirect damage to host cells.

### Example 6

In this Example, alloreactive T cell response to transplant conditioning and cytokine release was analyzed. Particularly, because the CD146CCR5 T-cell population circulates early post-HCT and endothelial CD146 is overexpressed during inflammation, it was believed that in response to transplant conditioning and cytokine release, alloreactive T cells would express the double-positive population in parallel to increased CD146 expression in the intestinal endothelium and increased CCL14 release from the epithelium. This would allow CD146CCR5 T cells to transmigrate across the endothelium via CD146-CD146 interaction and then to come in contact with the intestinal epithelium through chemotaxis to CCL14.

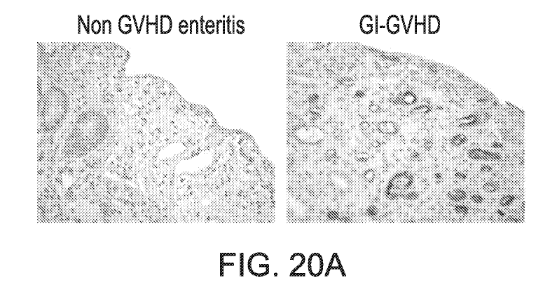

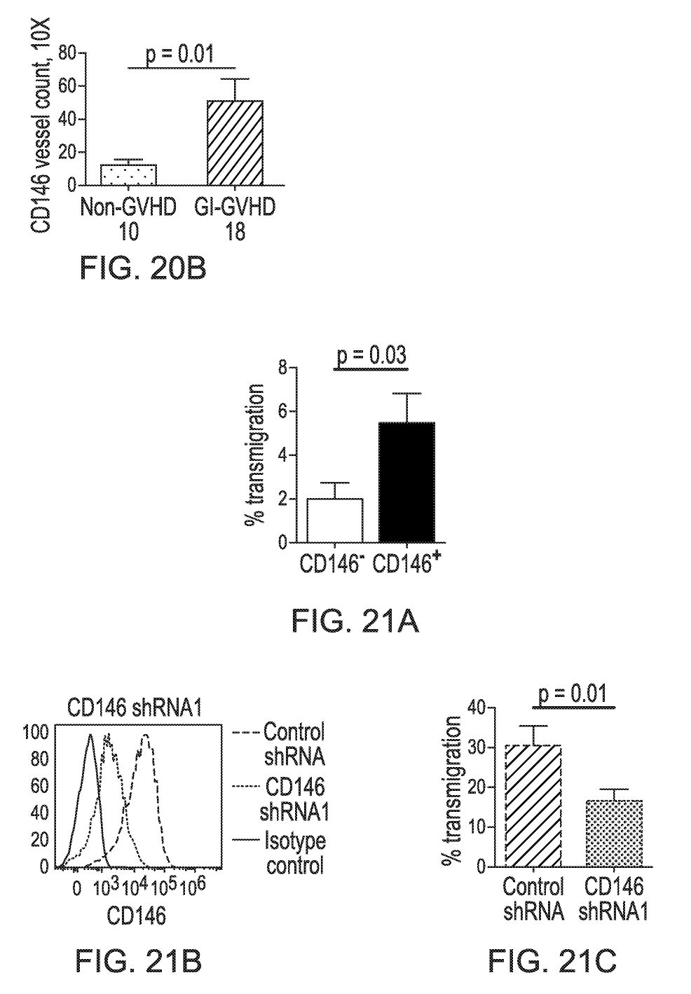

To test the hypothesis that CD146 is upregulated in GVHD-activated intestine, colonic biopsies of patients with non-GVHD enteritis and GI-GVHD were stained for CD146. Significantly greater CD146 expression was observed on the endothelium of GI-GVHD patients (FIGS. 20A & 20B and Table 3).

TABLE 3
CD146 on T cells and vessels in GI biopsies of GI-GVHD and non-
GVHD enteritis post-HCT

| | | | | CD146 |
|---|---|---|---|---|
| | GVHD | CD3 T | CD146 T | vessel count, |
| Patient | status | cells | cells | 10X |
| Patient 1 | GVHD | 220 | 0 | 0 |
| Patient 2 | GVHD | 67 | 3 | 32 |
| Patient 3 | GVHD | 97 | 0 | 6 |
| Patient 4 | GVHD | 58 | 0 | 105 |
| Patient 5 | GVHD | 55 | 3.3 | 8 |
| Patient 6 | GVHD | 90 | 1 | 82 |
| Patient 7 | GVHD | 48 | 0 | 14 |
| Patient 8 | GVHD | 42 | 2 | 9 |
| Patient 9 | GVHD | 137 | 2 | 9 |
| Patient 10 | GVHD | 97 | 4 | 24 |
| Patient 11 | GVHD | 156 | 3.3 | 229 |
| Patient 12 | GVHD | 86 | 2.7 | 56 |
| Patient 13 | GVHD | 43 | 0 | 46 |
| Patient 14 | GVHD | 364 | 4.7 | 86 |
| Patient 15 | GVHD | 157 | 3.3 | 82 |
| Patient 16 | GVHD | 100 | 3 | 82 |
| Patient 17 | GVHD | 68 | 7.7 | 36 |
| Patient 18 | GVHD | 34 | 3 | 13 |
| Patient 19 | Non-GVHD | 182 | 1 | 21 |
| Patient 20 | Non-GVHD | 19 | 1.3 | 13 |
| Patient 21 | Non-GVHD | 45 | 5.3 | 3 |
| Patient 22 | Non-GVHD | 31 | 0 | 7 |
| Patient 23 | Non-GVHD | 115 | 0 | 0 |
| Patient 24 | Non-GVHD | 68 | 0 | 0 |
| Patient 25 | Non-GVHD | 155 | 3 | 30 |
| Patient 26 | Non-GVHD | 86 | 8.3 | 23 |
| Patient 27 | Non-GVHD | 50 | 0 | 13 |
| Patient 28 | Non-GVHD | 55 | 1 | 14 |

These results suggest that both endothelial and lymphocytic CD146 could play an important role in recruiting pathogenic T cells to the intestine. If the hypothesis is true, more CD146⁺ and Th17-differentiated T cells should transmigrate through the activated endothelium as compared to CD146⁻ and Th1 cells. Indeed, more CD146⁺ or Th17 cells migrated through tumor necrosis factor-alpha-activated ECs in transwell assays (FIGS. 21A-21C).

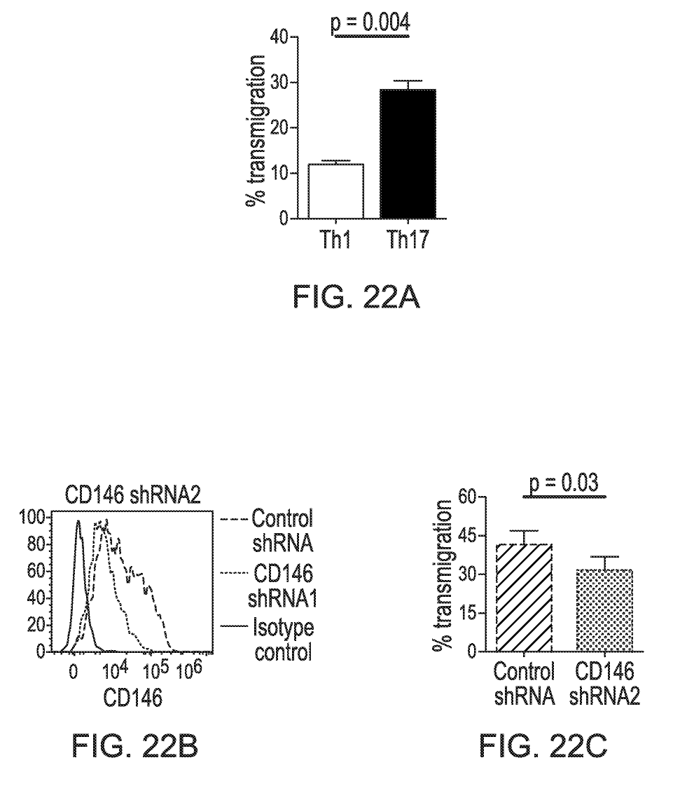

Lentivirally induced shRNA1 knockdown of CD146 in CD4 T cells led to a significant reduction in T-cell transmigration (FIG. 22A). To eliminate possible off-target effects, these results were verified with a second shRNA (FIGS. 22B & 25C). However, siRNA knockdown of CD146 on ECs did not reduce T-cell transmigration (not shown), suggesting that CD146 on T cells, but not on ECs, is a key promoter of pathogenic T-cell infiltration into GVHD target organs.

### Example 7

In this Example, the chemotaxis of CD146CCR5 T cells to CCL14 was evaluated. CCR5 on T cells can bind both CCL14 and CCL5, but only CCL14 was identified in the proteomics experiment. Thus, the chemotaxis of CD146CCR5 T cells to CCL14 and CCL14+CCL5 was tested.

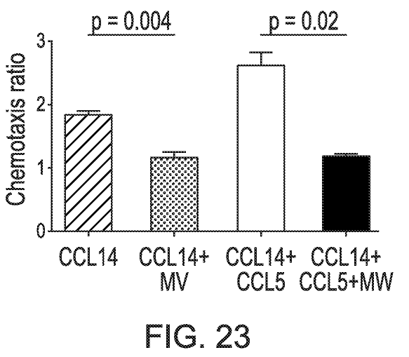

The CD146CCR5 T cells trafficking towards CCL14 and towards both CCL14+CCL5 was significantly increased and even more toward CCL14+CCL5. This chemotaxis was inhibited by the CCR5 inhibitor maraviroc (FIG. 23).

### Example 8

In this Example, the in vivo role of CD146 on T cells was evaluated.

CD146 knockout T cells in allogeneic murine GVHD models were used. Transfer of CD146 knockout T cells had not previously been used in immune-mediated disease models. The GVHD severity did not differ upon transplantation of these cells versus wild-type T cells (FIG. 27A-27D), which may be due to the low expression of CD146 on donor murine T cells compared to human T cells (FIG. 27E). Therefore, donor human T cells with shRNA-induced CD146 knockdown was used in a xenogeneic GVHD model.

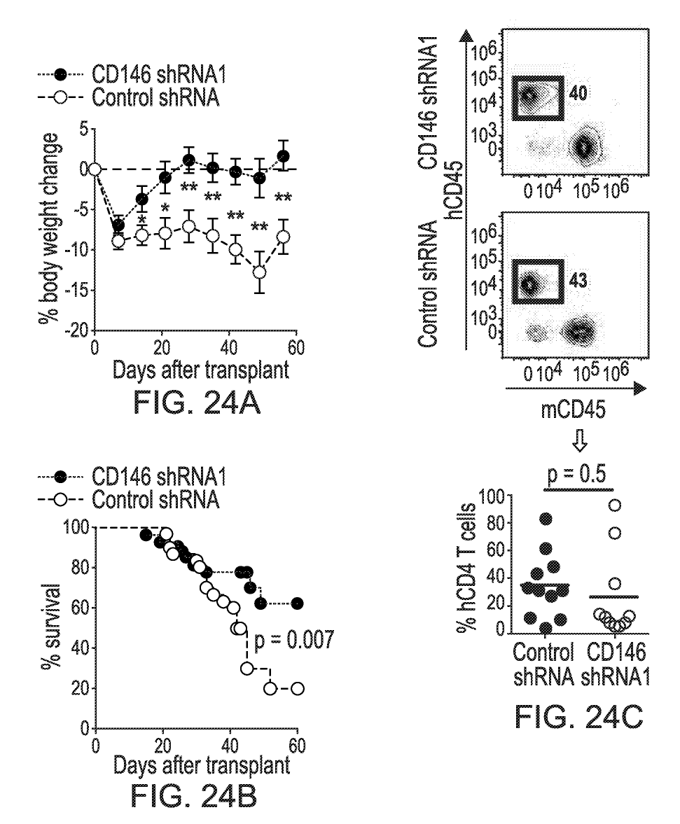

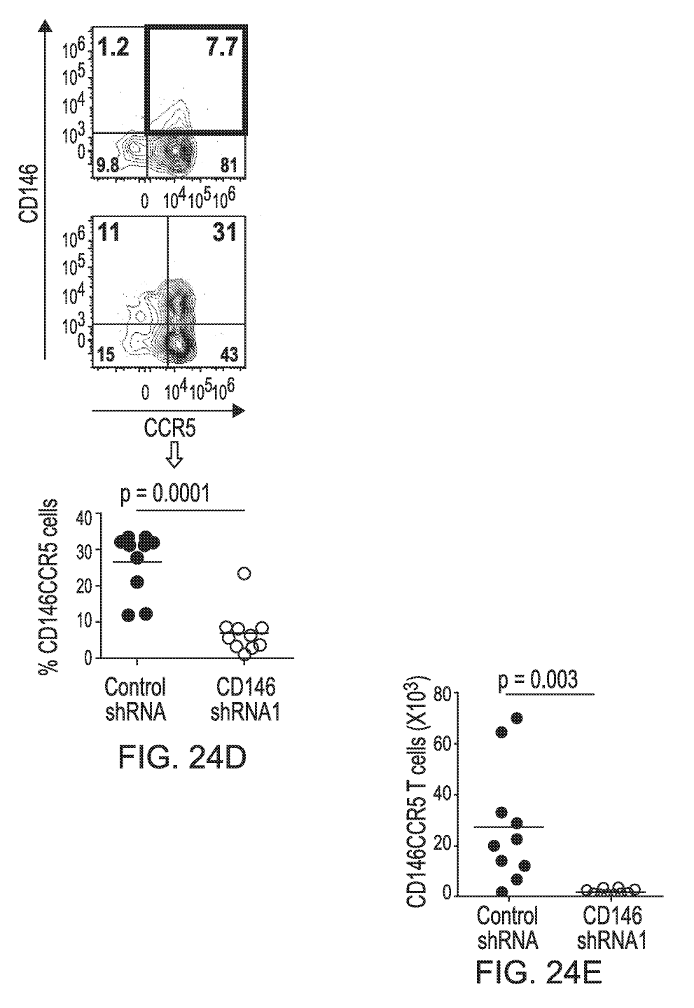

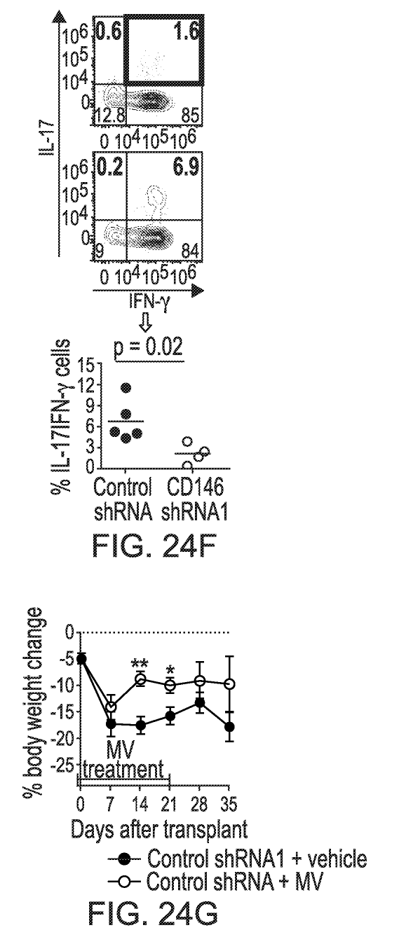

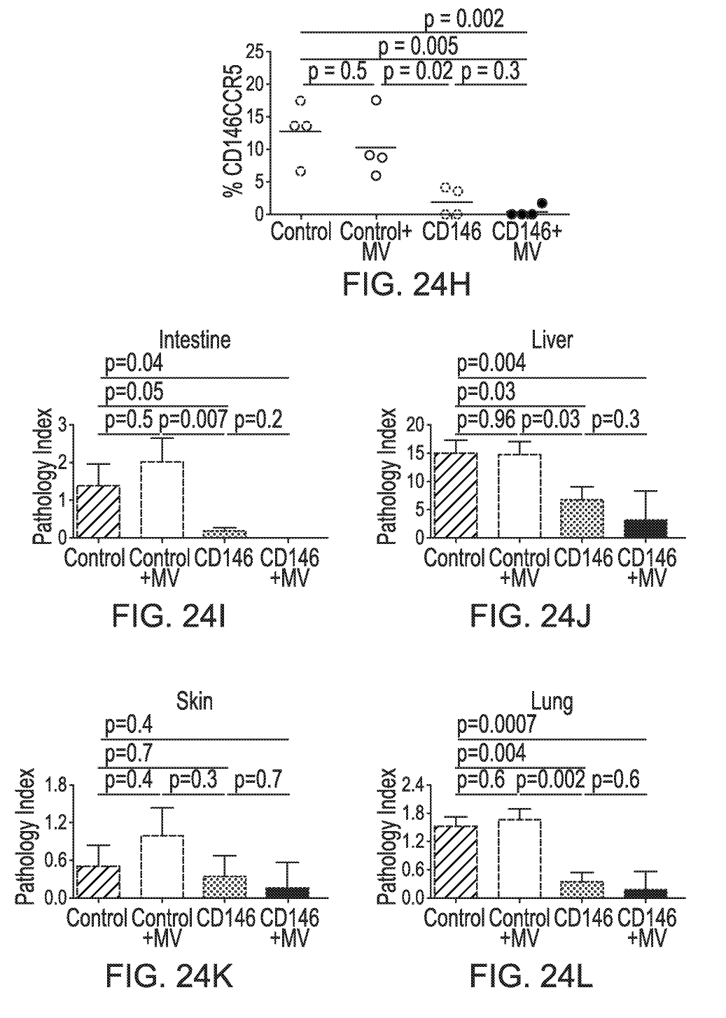

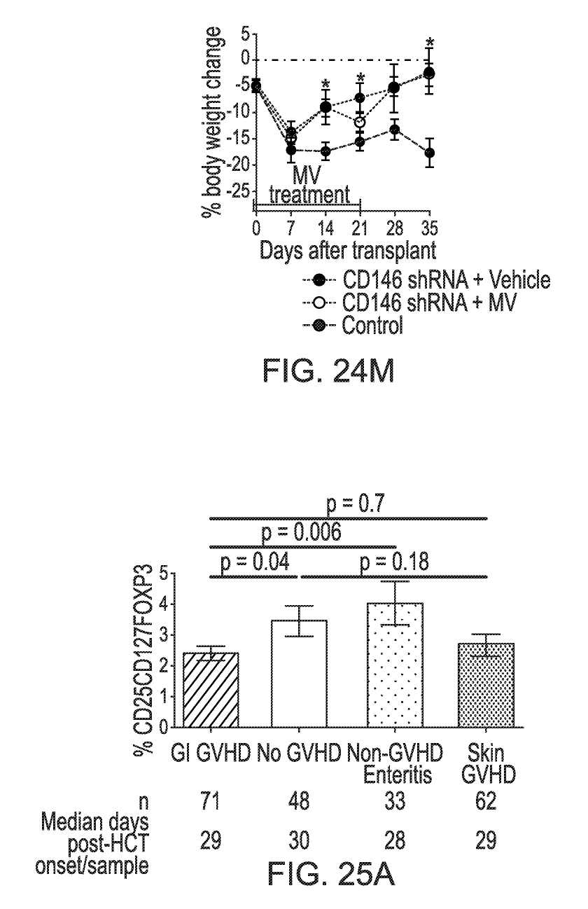

In comparison to the control group, immunodeficient NOD/scid/IL-2Rγ^{-/-} (NSG) mice transplanted with CD146 shRNA-transduced T cells did not lose weight (FIG. 24A), had better survival (FIG. 24B), showed similar human T-cell engraftment (FIG. 24C), had fewer CD146CCR5 T cells in the intestine (frequencies and absolute counts, FIGS. 24D and 24E, respectively), and had fewer IL-17/IFN-γ coexpressing T cells (FIG. 24F).

In order to test the role of CCR5 inhibition and because transfer of double CD146 and CCR5 shRNA-transduced T cells is not technically possible, the CCR5 inhibitor maraviroc was used in the xenogeneic model and compared its action to those of the control shRNA-transduced T cells and the CD146 shRNA-transduced T cells plus maraviroc. NSG mice transplanted with control shRNA-transduced T cells and treated with maraviroc for 21 days lost less weight than the untreated mice transplanted with control shRNA-transduced T cells (FIG. 24G), had less CD146CCR5 T-cell gut infiltration (FIG. 24H), and showed less severe pathology indexes of the intestine as well as liver, skin, and lung (FIGS. 24I-24L). In addition, there were no differences in bodyweight, CD146CCR5 T cell infiltration in the intestine, and pathology indexes between NSG mice transplanted with CD146 shRNA-transduced T cells and those transplanted with CD146 shRNA-transduced T cells and treated with maraviroc (FIGS. 24H-24M), possibly suggesting that CCR5 is acquired by the Th17-committed CD146 T cells during GVHD development.

### Example 9

In this Example, CD146CCR5 Treg frequency in GI-GVHD patients was analyzed.

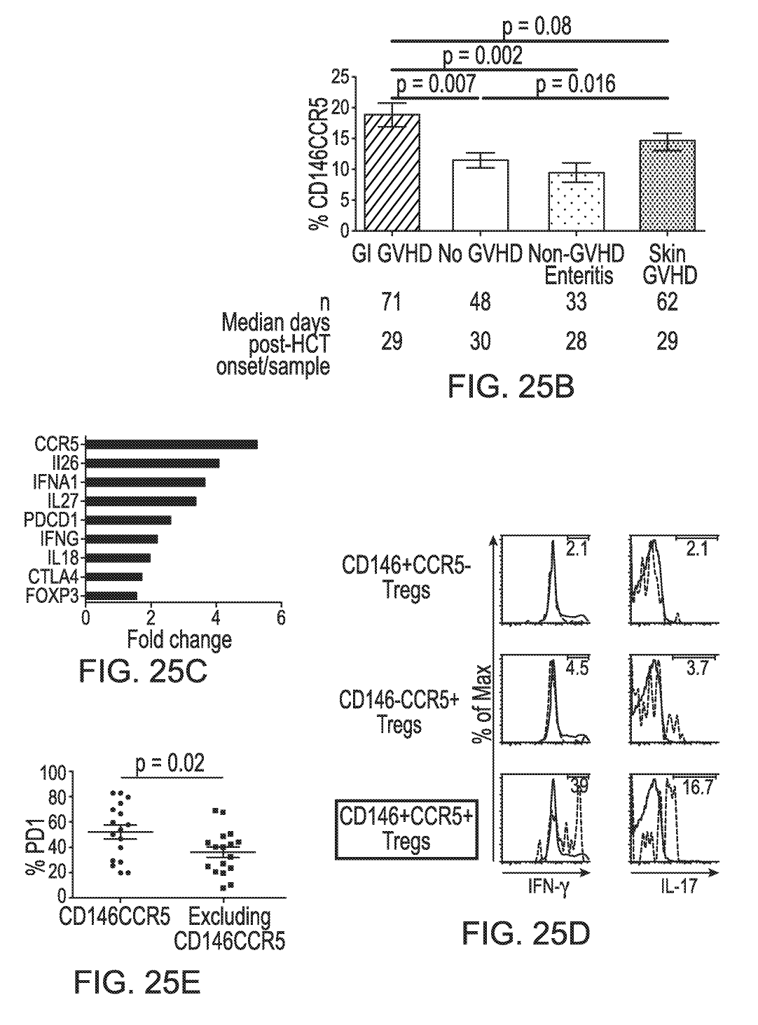

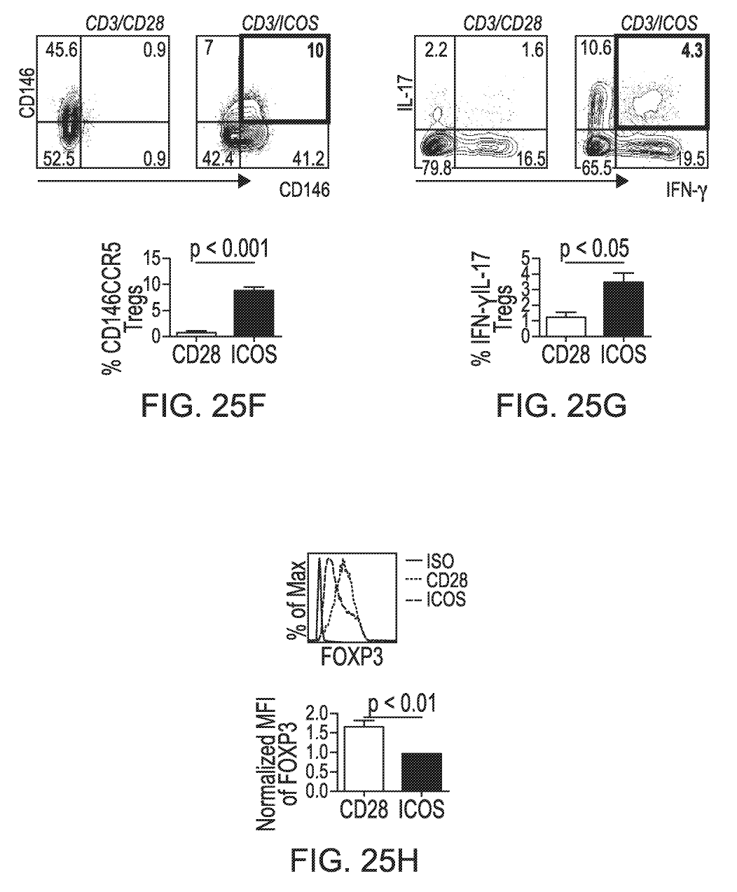

Regulatory T cells (Tregs) are crucial for the inhibition of GVHD development. In this Example, the frequency of total Tregs defined by CD25⁺CD127⁻FOXP3⁺ on CD4 T cells was significantly decreased in GI-GVHD patients compared to patients with non-GVHD enteritis (p=0.006) and patients without GVHD (p=0.04) (FIG. 25A). However, the frequency of CD146CCR5 Tregs was increased in GI-GVHD patients as compared to patients with non-GVHD enteritis (p=0.002) and patients without GVHD (p=0.007; FIG. 25B).

CD146CCR5 Tregs was further characterized using nanostring analysis and it was found that although they still express FOXP3, they also express inflammatory molecules such as IL-26, INF-α, IL-27, IFN-y, IL-18, and the exhaustion marker PD-1 (FIG. 25C). Expression of IFN-y, IL-17, and PD-1 in the double-positive population Tregs as compared to the Tregs excluding CD146CCR5 was confirmed at the protein level by flow cytometry (FIGS. 25D & 25E). These data suggest that the CD146CCR5 Tregs show increased plasticity toward Th17. To explore this hypothesis, human Tregs were differentiated in CD28 or ICOS conditions, and it was found that ICOS stimulation increased the frequency of CD146CCR5 T cells (FIG. 25F), increased the percentage of cells co-expressing IL-17/IFN-y (FIG. 25G) among the Tregs, and decreased the intensity of FOXP3 expression (FIG. 25H).

### DISCUSSION

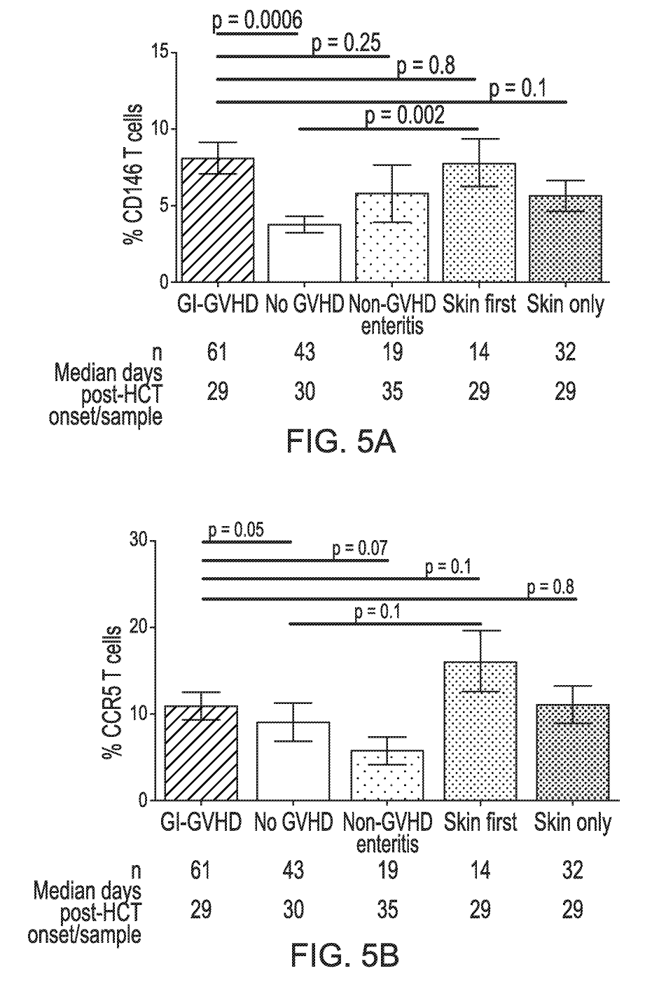

Progress toward clinical use of biomarkers requires the discovery, qualification, verification, optimization, and clinical validation of candidate markers before they can be incorporated into existing therapeutic diagnostic platforms. Earlier detection of aGVHD within a more treatable stage would be extremely valuable for improving outcomes in HCT patients. In the present disclosure, a mass spectrometry-based technique was used to unambiguously identify candidate plasma biomarkers of GI-GVHD, the primary cause of NRM after HCT. More particularly, a cellular CD146CCR5 T-cell population was identified based on the discovery of plasma ligands as a biomarker of GI-GVHD with diagnostic and prognostic value. CD146CCR5 T-cell frequencies were 4 times higher in patients with GI-GVHD than in patients with diarrhea who did develop GVHD (as confirmed later by colonic biopsies). The area under the ROC curve comparing CD146CCR5 T-cell frequencies between GI-GVHD and non-GVHD enteritis was 0.84. There were no differences in the frequencies of T cells expressing only CD146 or CCR5 between patients with GI-GVHD and non-GVHD enteritis. Indeed, as shown in FIGS. 5A & 5B, CCR5 seems to add to the model because it allows better differentiation of GI-GVHD and Non-GVHD enteritis (p=0.07), whereas CD146 single-positive CD4 T cells did not. The high frequency of CD146CCR5 T cells circulating before the occurrence of GI symptoms and as early as day 14 post-HCT suggests that their “expansion” is an early event in the pathology of GI-GVHD. Furthermore, as shown in FIG. 2C, the majority of patients with paired samples had stable or decreasing frequencies of CD146CCR5 T cells in the blood, which suggests these cells have already homed to the gut, hence decreasing in the blood between the two time points (14 days on average). The frequencies of CD146CCR5 T cells were not correlated with Reg3α and ST2 that have been previously linked to GI-GVHD development, suggesting that this new T-cell population might be an independent indicator of GI-GVHD, likely via a different biological mechanism. The ability to identify high-risk patients based on CD146CCR5 T cells in the blood soon after transplantation, before the development of GI-GVHD, may permit more stringent monitoring and application of preemptive interventions.

Because the CD146CCR5 T-cell population circulates in patients' blood early post-HCT, their clonality was assessed by deep TCR sequencing of T cells, and no significant differences were found between GI-GVHD and non-GVHD enteritis patients. In addition, the CD146⁺CCR5⁺ T cells had significantly higher clonality than did the double-negative cells, and no specific clone emerged from this limited analysis. The data further showed that when analyzed at the circulating PB level, classical reduced and full intensity conditioning with conventional HCT does not restrict repertoire recovery, even if future GI-GVHD will develop.

The cell adhesion molecule CD146 is a general endothelial cell marker, but it was found that its expression in GI-GVHD endothelium was highly upregulated, which is consistent with findings in other inflammatory GI diseases.

In summary, the early measurement of the discovered CD146CCR5 T-cell population in the blood allows for identification of patients at risk for GI-GVHD and thus facilitates preemptive intervention via personalized medicine. This ICOS-stimulated, Th17-committed CD146CCR5 T-cell subset reveals opportunities for potential therapeutic targets in GI-GVHD, such as CD146, ICOS, and RORC in addition to the already targetable CCR5 that has been shown to alleviate GVHD.

In view of the above, it will be seen that the several advantages of the disclosure are achieved and other advantageous results attained. As various changes could be made in the above methods without departing from the scope of the disclosure, it is intended that all matter contained in the above description and shown in the accompanying drawings shall be interpreted as illustrative and not in a limiting sense.

When introducing elements of the present disclosure or the various versions, embodiment(s) or aspects thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A method of diagnosing or of aiding diagnosis of acute graft-versus-host disease (aGVHD) in a subject receiving hematopoietic stem cell transplantation (HSCT), the method comprising:
measuring in a biological sample from the subject receiving HSCT the CD146⁺CCR5⁺ T cell frequency; and
comparing the CD146⁺CCR5⁺ T cell frequency of the subject receiving HSCT to a CD146⁺CCR5⁺ T cell frequency of a control subject not receiving HSCT; wherein an elevated CD146⁺CCR5⁺ T cell frequency of the subject receiving HSCT compared to the CD146⁺CCR5⁺ T cell frequency of the control subject is indicative of aGVHD.

2. The method of claim 1 wherein the biological sample is measured using a method selected from the group consisting of immunohistochemistry, flow cytometry, transmigration assay, and combinations thereof.

3. The method of claim 1 wherein the biological sample is blood.

4. The method of claim 1 wherein the biological sample is measured prior to the onset of clinical symptoms of aGVHD.

5. The method of claim 1 wherein the control subject can be selected from a healthy individual, a patient receiving uncomplicated allogenic transplant, and a patient with non-GVHD enteritis.

6. The method of claim 1 wherein the subject receiving HSCT has or is susceptible to gastrointestinal GVHD (GI GVHD).

7. (canceled)

8. A method of prognosing or of aiding prognosis of acute graft-versus-host disease (aGVHD) in a subject receiving hematopoietic stem cell transplantation (HSCT), the method comprising:
measuring in a biological sample from the subject receiving HSCT the CD146+CCR5+ T cell frequency; and
comparing the CD146⁺CCR5⁺ T cell frequency of the subject receiving HSCT to a CD146⁺CCR5⁺ T cell frequency of a control subject not receiving HSCT; wherein an elevated CD146⁺CCR5⁺ T cell frequency of the subject receiving HSCT compared to the CD146⁺CCR5⁺ T cell frequency of the control subject is indicative of aGVHD.

9. The method of claim 8 wherein the biological sample is measured using a method selected from the group consisting of immunohistochemistry, flow cytometry, transmigration assay, and combinations thereof.

10. The method of claim 8 wherein the biological sample is blood.

11. The method of claim 8 wherein the biological sample is measured prior to the onset of clinical symptoms of aGVHD.

12. The method of claim 8 wherein the control subject can be selected from a healthy individual, a patient receiving uncomplicated allogenic transplant, and a patient with non-GVHD enteritis.

13. The method of claim 8 wherein the subject receiving HSCT has or is susceptible to gastrointestinal GVHD (GI GVHD).

14. (canceled)

15. A method of measuring treatment efficacy in an aGVHD subject, the method comprising:
measuring in a first biological sample from the subject a baseline CD146⁺CCR5⁺ T cell frequency;
administering a treatment for aGVHD; and
measuring in a second biological sample from the subject a post-treatment CD146⁺CCR5⁺ T cell frequency; wherein a post-treatment CD146⁺CCR5⁺ T cell frequency that is equal to or greater than the baseline CD146⁺CCR5⁺ T cell frequency is indicative of treatment inefficacy.

16. The method of claim 15 wherein the first biological sample and the second biological sample is measured using a method selected from the group consisting of immunohistochemistry, flow cytometry, transmigration assay, and combinations thereof.

17. The method of claim 15 wherein at least the first biological sample and the second biological sample is blood.

18. The method of claim 15 wherein at least the first biological sample is measured prior to the onset of clinical symptoms of aGVHD.

19. The method of claim 15 wherein the treatment for aGVHD is an immunosuppressant agent.

20. The method of claim 19 wherein the immunosuppressant agent is a steroid.

21. A diagnostic biomarker panel comprising a Th17-prone CD146⁺CCR5⁺ T-cell population and suppressor of tumorigenicity 2 (ST2).

22. A prognosis biomarker panel comprising a Th17-prone CD146⁺CCR5⁺ T-cell population and suppressor of tumorigenicity 2 (ST2).
